(19) United States
(12) Patent Application Publication
(10) Pub. No.: US 2025/0228840 A1
(43) Pub. Date: Jul. 17, 2025

(54) METHODS OF TREATING SJÖGREN-LARSSEN SYNDROME

(71) Applicant: Aldeyra Therapeutics, Inc., Lexington, MA (US)

(72) Inventors: Todd Brady, Carlisle, MA (US); Adam Brockman, Arlington, MA (US); Stephen Gitu Machatha, Wilmington, MA (US)

(21) Appl. No.: 18/850,826

(22) PCT Filed: Mar. 29, 2023
(86) PCT No.: PCT/US2023/016713

§ 371 Date: Sep. 25, 2024

Related U.S. Application Data
(60) Provisional application No. 63/362,116, filed on Mar. 29, 2022.

(51) Int. Cl. A61K 31/47; A61K 9/00; A61P 3/00

(52) U.S. Cl. CPC A61K 31/47; A61K 9/0053; A61P 3/00

(57) ABSTRACT

The present invention relates to methods of treatment using a quinoline compound, or a pharmaceutically acceptable salt thereof, for treatment of a disease, disorder, or condition such as Sjögren-Larsson syndrome (SLS).

### CROSS-REFERENCE TO RELATED APPLICATIONS

Tis application claims the benefit of priority to U.S. Provisional Patent Application Ser. No. 63/362,116, filed Mar. 29, 2022, the entirety of which is hereby incorporated by reference.

### TECHNICAL FIELD

The present invention provides methods of treating Sjögren-Larsson syndrome (SLS) using small molecule aldehyde trapping compounds.

### BACKGROUND

The buildup or improper processing of toxic aldehyde species in the body underlies or is implicated in neurological, autoimmune, and inflammatory conditions, such as skin, lung, ocular, and systemic conditions. Metabolic and inflammatory processes in cells generate toxic aldehydes, such as malondialdehyde (MDA), 4-hydroxyl-2-nonenal (4-HNE), glyoxal, and methylglyoxal. These aldehydes are highly reactive with proteins, carbohydrates, lipids and DNA, leading to chemically modified biological molecules, activation of inflammatory mediators such as NF-kappa B, and damage in diverse organs. For example, retinaldehyde can react with phosphatidylethanolamine (PE) to form a highly toxic compound called A2E, which is a component of lipofuscin that is believed to be involved in the development and progression of Age-Related Macular Degeneration (AMD). Many bodily defense mechanisms function to remove or lower the levels of toxic aldehydes, including metabolism by aldehyde dehydrogenases, buffering by molecules such as glutathione (GSH) and removal from sites of potential toxicity by transporters such as ABCA4. Novel small molecule therapeutics can be used to scavenge “escaped” retinaldehyde in the retina, thus reducing A2E formation and lessening the risk of AMD (Jordan et al. (2006)).

Aldehydes are implicated in diverse pathological conditions such as dry eye, cataracts, keratoconus, Fuchs' endothelial dystrophy in the cornea, uveitis, allergic conjunctivitis, ocular cicatricial pemphigoid, conditions associated with photorefractive keratectomy (PRK) healing or other corneal healing, conditions associated with tear lipid degradation or lacrimal gland dysfunction, inflammatory ocular conditions such as ocular rosacea (with or without meibomian gland dysfunction), and non-ocular disorders or conditions such as skin cancer, psoriasis, contact dermatitis, atopic dermatitis, acne vulgaris, and Sjögren-Larsson Syndrome. Sjögren-Larsson syndrome (SLS) is an inherited disorder of fatty aldehyde metabolism characterized by clinical features of congenital ichthyosis, spastic diplegia, intellectual disability, seizures, and a distinctive retinopathy. The disease is caused by biallelic mutations in ALDH3A2, which results in deficient activity of fatty aldehyde dehydrogenase (FALDH) and leads to the build-up of harmful long-chain (C16-C20) aldehydes and alcohols. Accumulation of these lipids and their metabolic products in skin, brain, and eyes is responsible for the symptoms. The debilitating symptoms persist life-long, and there is no FDA-approved drug for SLS that addresses the pathogenic mechanisms.

Aldehydes are toxic molecules due to their propensity to covalently attach to proteins and other target molecules containing certain reactive chemical groups. Drugs that detoxify reactive aldehydes are under development for various inflammatory conditions characterized by the excessive formation of aldehydes. Aldehydes produced during inflammation typically have short- or medium-chain lengths (C3-C9), which do not accumulate in SLS. There is a need for effective treatments to reduce the formation of long-chain (C16-C18) fatty aldehyde adducts with cellular lipids, mitigate aldehyde cytotoxicity, and prevent metabolism of fatty aldehydes to fatty alcohols. There is also a need for effective treatments to improve the other symptoms of SLS, such as developmental delay, spasticity, and neurological symptoms.

The present invention addresses this need and provides other related advantages.

### SUMMARY

In one aspect, the present disclosure provides a method of treating a disease, disorder, or condition selected from hidradenitis suppurativa, toxoplasmosis, Sjögren-Larsson syndrome (SLS), Severe Premenstrual Syndrome (severe PMS), premenstrual dysphoric disorder (PMDD), obsessive-compulsive disorder (OCD), bipolar disorder, depression, major depressive disorder (MDD), or Posttraumatic Stress Disorder (PTSD), comprising administering to a subject in need thereof an effective amount of a quinoline compound described herein, or a pharmaceutically acceptable salt thereof.

In one aspect, the present disclosure provides a method of treating a disease, disorder, or condition selected from hidradenitis suppurativa, toxoplasmosis, Sjögren-Larsson syndrome (SLS), Severe Premenstrual Syndrome (severe PMS), premenstrual dysphoric disorder (PMDD), obsessive-compulsive disorder (OCD), bipolar disorder, depression, major depressive disorder (MDD), or Posttraumatic Stress Disorder (PTSD), comprising administering to a subject in need thereof an effective amount of Compound 1:

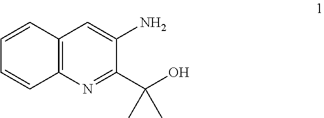

or a pharmaceutically acceptable salt thereof.

In one aspect, the present invention provides a method of treating Sjögren-Larsson syndrome (SLS), comprising administering to a patient in need thereof an effective amount of Compound 1:

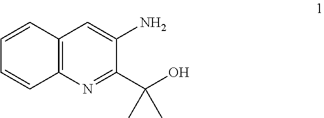

or a pharmaceutically acceptable salt thereof.

In some embodiments, Compound 1 or a pharmaceutically acceptable salt thereof is administered at a dose of about 100 mg to about 600 mg per day. In some embodiments, Compound 1 or a pharmaceutically acceptable salt thereof is administered at a dose of about 250 mg per day. In some embodiments, Compound 1 or a pharmaceutically acceptable salt thereof is administered at a dose of about 100, 125, 150, 175, 200, 225, 250, 275, 300, 325, 350, 375, 400, 425, 450, 475, 500, 525, 550, 575, or 600 mg per day.

In some embodiments, Compound 1 or a pharmaceutically acceptable salt thereof is administered systemically. Compound 1 or a pharmaceutically acceptable salt thereof is administered orally.

In some embodiments, the method improves one or more symptoms of SLS. In some embodiments, the method improves pruritis or ichthyosis. In some embodiments, the method improves myopia, photophobia, and macular degeneration. In some embodiments, the method improves seizures, spasticity, cognitive delay, neurological symptoms, or neurodegeneration associated with SLS.

Unit dosage forms comprising Compound 1 or a pharmaceutically acceptable salt thereof are suitable for administration to a patient for treating, ameliorating, or preventing diseases, disorders, and conditions described herein, such as Sjögren-Larsson syndrome (SLS).

In some embodiments, a unit dosage form of the invention comprises about 5 mg to about 600 mg of the quinoline compound such as I-1, or a pharmaceutically acceptable salt thereof. In some embodiments, a unit dosage form of the invention is suitable for oral administration. In some embodiments, a unit dosage form of the invention comprises one or more pharmaceutically acceptable excipients or carriers. In some embodiments, the one or more pharmaceutically acceptable excipients or carriers are selected from one or more of Eudragit L100, microcrystalline cellulose, lactose monohydrate, croscarmellose sodium, crospovidone (Kollidon CL), a vinylpyrrolidone-vinyl acetate copolymer such as Kollidon VA64, sodium lauryl sulfate, and magnesium stearate. In some embodiments, a unit dosage form of the invention exhibits pharmacokinetics results as described herein.

In another aspect, the present invention provides a method for reducing levels of one or more toxic aldehydes in a subject, comprising administering to a subject in need thereof a pharmaceutical composition, as described herein.

In some embodiments, the toxic aldehyde is selected from formaldehyde, acetaldehyde, acrolein, glyoxal, methylglyoxal, hexadecanal, octadecanal, hexadecenal, succinic semi-aldehyde, malondialdehyde, 4-hydroxynonenal, 4-hydroxy-2E-hexenal, 4-hydroxy-2E,6Z-dodecadienal, retinaldehyde, leukotriene B4 aldehyde, and octadecenal.

In some embodiments, the toxic aldehyde is formaldehyde. In some embodiments, the toxic aldehyde is acetaldehyde. In some embodiments, the toxic aldehyde is acrolein. In some embodiments, the toxic aldehyde is glyoxal. In some embodiments, the toxic aldehyde is methylglyoxal. In some embodiments, the toxic aldehyde is hexadecanal. In some embodiments, the toxic aldehyde is octadecanal. In some embodiments, the toxic aldehyde is hexadecenal. In some embodiments, the toxic aldehyde is succinic semi-aldehyde (SSA). In some embodiments, the toxic aldehyde is malondialdehyde (MDA). In some embodiments, the toxic aldehyde is 4-hydroxynonenal. In some embodiments, the toxic aldehyde is retinaldehyde. In some embodiments, the toxic aldehyde is 4-hydroxy-2E-hexenal. In some embodiments, the toxic aldehyde is 4-hydroxy-2E,6Z-dodecadienal. In some embodiments, the aldehyde is leukotriene B4 aldehyde. In some embodiments, the aldehyde is octadecenal.

In some embodiments, the unit dosage form is administered systemically.

In some embodiments, the unit dosage form is administered orally.

### DETAILED DESCRIPTION

### 1. General Description of Certain Aspects of the Invention

In some aspects, the present disclosure provides methods of treating a disease, disorder, or condition described herein. In some aspects, the present disclosure provides compounds, compositions, and methods for the treatment, amelioration, prevention, and/or reduction of a risk of a disease, disorder, or condition such as those described herein.

In one aspect, the present disclosure provides a method of treating a disease, disorder, or condition selected from hidradenitis suppurativa, toxoplasmosis, Sjögren-Larsson syndrome (SLS), Severe Premenstrual Syndrome (PMS), premenstrual dysphoric disorder (PMDD), obsessive-compulsive disorder (OCD), Bipolar Disorder, depression, major depressive disorder (MDD), or Posttraumatic Stress Disorder (PTSD), comprising administering to a subject in need thereof an effective amount of a quinoline compound, such as a compound of Formula I:

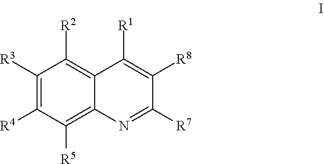

or a pharmaceutically acceptable salt thereof, wherein:
each of R¹, R⁷, and R⁸ is independently H, D, halogen, —NH₂, —CN, —OR, —SR, optionally substituted C₁₋₆ aliphatic, or

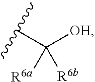

wherein one of R¹, R⁷, and R⁸ is —NH₂ and one of R¹ R⁷, and R⁸ is

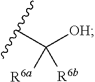

R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R^{6a} is C₁₋₄ aliphatic optionally substituted with 1, 2, or 3 deuterium or halogen atoms;
R^{6b} is C₁₋₄ aliphatic optionally substituted with 1, 2, or 3 deuterium or halogen atoms; or R^{6a} and R^{6b}, taken together with the carbon atom to which they are attached, form a 3- to 8-membered cycloalkyl or heterocyclyl ring containing 1-2 heteroatoms selected from nitrogen, oxygen, and sulfur; and
each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In another aspect, the compound is of formula II:

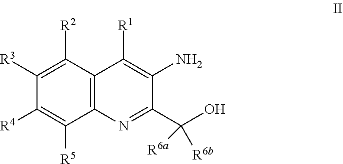

or a pharmaceutically acceptable salt thereof, wherein:
R¹ is H, D, or halogen;
R² is H, D, or halogen;
R³ is H, D, or halogen;
R⁴ is H, D, or halogen;
R⁵ is H, D, or halogen;
R^{6a} is C₁₋₄ aliphatic optionally substituted with 1, 2, or 3 deuterium or halogen atoms; and
R^{6b} is C₁₋₄ aliphatic optionally substituted with 1, 2, or 3 deuterium or halogen atoms.

### 2. Definitions

Compounds of the present invention include those described generally above, and are further illustrated by the classes, subclasses, and species disclosed herein. As used herein, the following definitions shall apply unless otherwise indicated. For purposes of the present disclosure, the chemical elements are identified in accordance with the Periodic Table of the Elements, CAS version, Handbook of Chemistry and Physics, 75^{th} Ed. Additionally, general principles of organic chemistry are described in Organic Chemistry, Thomas Sorrell, University Science Books, Sausalito: 1999, and March's Advanced Organic Chemistry, 5^{th} Ed., Ed.: Smith, M. B. and March, J., John Wiley & Sons, New York: 2001, the entire contents of which are hereby incorporated by reference.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen; or a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In some embodiments, the term “aryl” used alone or as part of a larger moiety as in “aralkyl,” “aralkoxy,” or “aryloxyalkyl,” refers to monocyclic and bicyclic ring systems having a total of five to 10 ring members, wherein at least one ring in the system is aromatic and wherein each ring in the system contains three to seven ring members. In certain embodiments of the compounds, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 □ electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned for the compounds herein are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen, —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C_{1- 4} straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5- to 6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5 to 6-membered saturated, partially unsaturated, or aryl ring having 0-4 heteroatoms independently selected from nitrogen, oxygen, and sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5- to 6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, and sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, mesylate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

As used herein, the terms “fasting state,” “fasting condition,” “fasted state,” or “fasted condition,” refer to a state or condition following at least about 8 hour fast from all food and drink (except water). In some embodiments, a subject is to fast from all food and drink (except water) for at least 8 hours prior to receiving dosing in the morning, and continue to fast until lunch.

As used herein, the terms “fed state” or “fed condition” refer to a state or condition following a standardized high-fat meal. In some embodiments, an administration in a fed state refers to an administration following a standard FDA high-fat breakfast. In some embodiments, subjects receiving the fed regimen fast from all food and drink (except water) for at least 8 hours prior to receiving a test meal. In some embodiments, prior to dosing on Day 1, subjects are served a standard FDA high-fat breakfast composed of 2 eggs fried in butter, 2 strips of bacon, 2 slices of toast with 2 pats of butter, 4 ounces of hash browns and 8 ounces of whole milk, to be ingested and completely consumed within 20 minutes. This breakfast contains approximately 150 protein kcal, 250 carbohydrate kcal, and 500-600 fat kcal. In some embodiments, an alternative meal may be given provided the meal has similar composition and caloric contents.

As used herein, “about” or “approximately” in reference to a numerical value means that the stated numerical value may vary by up to 10% of the stated value. For example, “about 10” refers to a value of 9.9 to 10.1 (10+/−0.1).

### 3. Detailed Description of Embodiments

The compounds described herein are quinoline compounds that have aldehyde trapping activity, and have been described for use in treating disorders and diseases associated with the effects of toxic aldehydes. See, e.g., PCT patent publication WO2006127945, WO2014116836, WO2017035077, and WO2017035082, each of which is hereby incorporated by reference. Synthesis of the compounds herein are described in PCT publications WO2006127945, WO2017035082, and WO2018039192; and U.S. patent application publication US 2013/0190500, each of which is hereby incorporated by reference. As described in the present disclosure, certain quinoline compounds are useful in treating a disease, disorder, or condition described herein.

In addition, the disclosures of the following patent applications are hereby incorporated by reference: WO 2019/075136, filed Oct. 10, 2018; WO2021/195211; and WO2021/231792. These applications provide additional disclosure related to the quinoline compounds described herein, including their use in treating certain diseases.

In one aspect, the present disclosure provides a method of treating a disease, disorder, or condition selected from hidradenitis suppurativa, toxoplasmosis, Sjögren-Larsson syndrome (SLS), Severe Premenstrual Syndrome (severe PMS), premenstrual dysphoric disorder (PMDD), obsessive-compulsive disorder (OCD), bipolar disorder, depression, major depressive disorder (MDD), and Posttraumatic Stress Disorder (PTSD), comprising administering to a subject in need thereof an effective amount of a compound of Formula I:

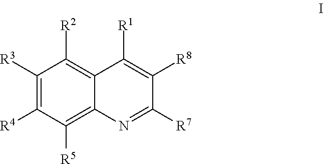

or a pharmaceutically acceptable salt thereof, wherein:
each of R¹, R⁷, and R⁸ is independently H, D, halogen, —NH₂, —CN, —OR, —SR, optionally substituted C₁₋₆ aliphatic, or

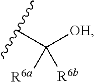

wherein one of R¹, R⁷, and R⁸ is —NH₂ and one of R¹ R⁷, and R⁸ is

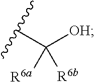

R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R^{6a} is C₁₋₄ aliphatic optionally substituted with 1, 2, or 3 deuterium or halogen atoms;
R^{6b} is C₁₋₄ aliphatic optionally substituted with 1, 2, or 3 deuterium or halogen atoms; or R^{6a} and R^{6b}, taken together with the carbon atom to which they are attached, form a 3- to 8-membered cycloalkyl or heterocyclyl ring containing 1-2 heteroatoms selected from nitrogen, oxygen, and sulfur; and
each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In another aspect, the compound is of formula II:

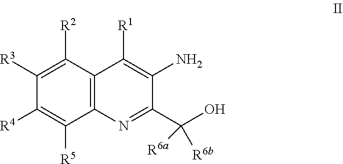

or a pharmaceutically acceptable salt thereof, wherein:
R¹ is H, D, or halogen;
R² is H, D, or halogen;
R³ is H, D, or halogen;
R⁴ is H, D, or halogen;
R⁵ is H, D, or halogen;
R^{6a} is C₁₋₄ aliphatic optionally substituted with 1, 2, or 3 deuterium or halogen atoms; and
R^{6b} is C₁₋₄ aliphatic optionally substituted with 1, 2, or 3 deuterium or halogen atoms.

In some embodiments, the compound is Compound 1:

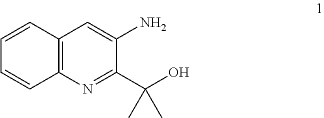

or a pharmaceutically acceptable salt thereof.

In some embodiments, the disease, disorder, or condition is hidradenitis suppurativa. In some embodiments, the disease, disorder, or condition is toxoplasmosis. In some embodiments, the disease, disorder, or condition is Sjögren-Larsson syndrome (SLS). In some embodiments, the disease, disorder, or condition is Severe Premenstrual Syndrome (severe PMS). In some embodiments, the disease, disorder, or condition is premenstrual dysphoric disorder (PMDD). In some embodiments, the disease, disorder, or condition is obsessive-compulsive disorder (OCD). In some embodiments, the disease, disorder, or condition is bipolar disorder. In some embodiments, the disease, disorder, or condition is depression. In some embodiments, the disease, disorder, or condition is major depressive disorder (MDD). In some embodiments, the disease, disorder, or condition is Posttraumatic Stress Disorder (PTSD).

In one aspect, the present invention provides a method of treating Sjögren-Larsson syndrome (SLS), comprising administering to a patient in need thereof an effective amount of Compound 1:

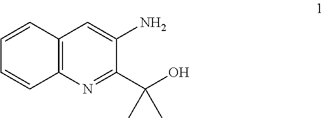

or a pharmaceutically acceptable salt thereof.

In some embodiments, Compound 1 or a pharmaceutically acceptable salt thereof is administered at a dose of about 100 mg to about 600 mg per day. In some embodiments, Compound 1 or a pharmaceutically acceptable salt thereof is administered at a dose of about 250 mg per day. In some embodiments, Compound 1 or a pharmaceutically acceptable salt thereof is administered at a dose of about 100, 125, 150, 175, 200, 225, 250, 275, 300, 325, 350, 375, 400, 425, 450, 475, 500, 525, 550, 575, or 600 mg per day.

In some embodiments, Compound 1 or a pharmaceutically acceptable salt thereof is administered systemically. Compound 1 or a pharmaceutically acceptable salt thereof is administered orally.

In some embodiments, the method improves one or more symptoms of SLS. In some embodiments, the method improves pruitis or ichthyosis. In some embodiments, the method improves myopia, photophobia, and macular degeneration. In some embodiments, the method improves spasticity, cognitive delay, neurological symptoms, or neurodegeneration associated with SLS.

The following embodiments are applicable to Formula I.

In some embodiments of Formula I, R^{6a} is C₁₋₄ aliphatic. In some embodiments, R^{6a} is C₁₋₄ aliphatic optionally substituted with 1, 2, or 3 deuterium atoms. In some embodiments, R^{6a} is C₁₋₄ aliphatic optionally substituted with 1, 2, or 3 halogen atoms.

In some embodiments of Formula I, R^{6a} is C₁₋₄ alkyl. In some embodiments, R^{6a} is C₁₋₄ alkyl optionally substituted with 1, 2, or 3 deuterium or halogen atoms. In some embodiments, R^{6a} is C₁₋₄ alkyl optionally substituted with 1, 2, or 3 halogen atoms. In some embodiments, R^{6a} is methyl or ethyl optionally substituted with 1, 2, or 3 halogen atoms. In some embodiments, R^{6a} is methyl.

As defined generally above, R^{6b} is C₁₋₄ aliphatic optionally substituted with 1, 2, or 3 deuterium or halogen atoms.

In some embodiments of Formula I, R^{6b} is C₁₋₄ aliphatic. In some embodiments, R^{6b} is C₁₋₄ aliphatic optionally substituted with 1, 2, or 3 deuterium atoms. In some embodiments, R^{6b} is C₁₋₄ aliphatic optionally substituted with 1, 2, or 3 halogen atoms.

In some embodiments of Formula I, R^{6b} is C₁₋₄ alkyl. In some embodiments, R^{6b} is C₁₋₄ alkyl optionally substituted with 1, 2, or 3 deuterium or halogen atoms. In some embodiments, R^{6b} is C₁₋₄ alkyl optionally substituted with 1, 2, or 3 halogen atoms. In some embodiments, R^{6b} is methyl or ethyl optionally substituted with 1, 2, or 3 halogen atoms. In some embodiments, R^{6b} is methyl.

As defined generally above, in some embodiments, R^{6a} and R^{6b}, taken together with the carbon atom to which they are attached, form a 3- to 8-membered cycloalkyl or heterocyclyl ring containing 1-2 heteroatoms selected from nitrogen, oxygen, and sulfur.

In some embodiments of Formula I, R^{6a} and R^{6b}, taken together with the carbon atom to which they are attached, form a 3- to 8-membered cycloalkyl. In some embodiments, R^{6a} and R^{6b}, taken together with the carbon atom to which they are attached, form a 3- to 8-membered heterocyclyl ring containing 1-2 heteroatoms selected from nitrogen, oxygen, and sulfur.

In some embodiments of Formula I, R^{6a} and R^{6b}, taken together with the carbon atom to which they are attached, form a cyclopropyl, cyclobutyl, or cyclopentyl ring. In some embodiments, R^{6a} and R^{6b}, taken together with the carbon atom to which they are attached, form an oxirane, oxetane, tetrahydrofuran, or aziridine.

In some embodiments of Formula I, the —NH₂ on one of R¹, R⁷, and R⁸ and the carbinol on the other of R¹, R⁷, and R⁸ are on adjacent carbon atoms of the pyridine moiety.

In some embodiments, the compound is a compound of Formula I-a, I-b, or I-c:

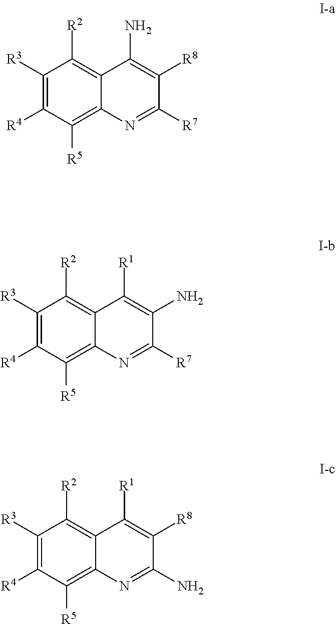

or a pharmaceutically acceptable salt thereof, wherein:
each of R¹, R⁷, and R⁸ when present is independently H, D, halogen, —CN, —OR, —SR, optionally substituted C₁₋₆ aliphatic, or

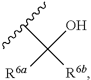

wherein one of R¹, R⁷, and R⁸ is

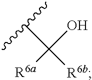

and
R², R³, R⁴, R⁵, R^{6a}, R^{6b}, R⁷, R⁸, and R are as defined for Formula I.

In some embodiments, the compound for use in the method is a compound of Formula I-d, I-e, I-f or I-g:

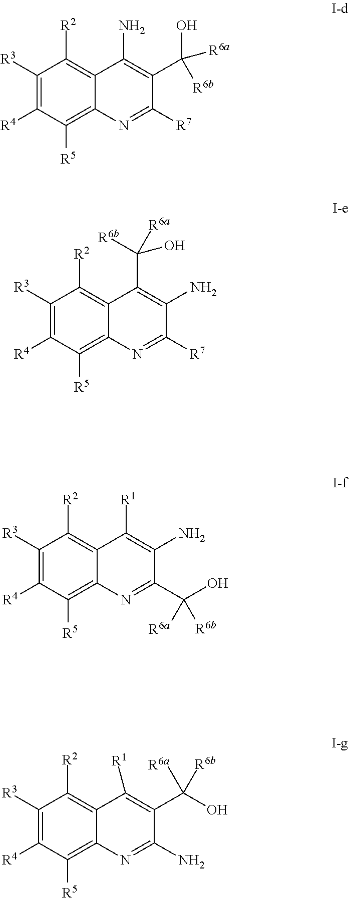

or a pharmaceutically acceptable salt thereof, wherein;
R¹ and R⁷ is independently H, D, halogen, —CN, —OR, —SR, optionally substituted C₁₋₆ aliphatic; and
R², R³, R⁴, R³, R^{6a}, R^{6b}, R⁷, R⁸, and R are as defined for Formula I.

The following embodiments are applicable to Formula H.

As defined generally above, R¹ is H, D, or halogen.

In some embodiments, R¹ is H. In some embodiments, R¹ is D. In some embodiments, R¹ is halogen. In some embodiments, R¹ is Cl. In some embodiments, R¹ is Br.

As defined generally above, R² is H, D, or halogen.

In some embodiments, R² is H. In some embodiments, R² is D. In some embodiments, R² is halogen. In some embodiments, R² is Cl. In some embodiments, R² is Br.

As defined generally above, R³ is H, D, or halogen.

In some embodiments, R³ is H. In some embodiments, R³ is D. In some embodiments, R³ is halogen. In some embodiments, R³ is Cl. In some embodiments, R³ is Br.

As defined generally above, R⁴ is H, D, or halogen.

In some embodiments, R⁴ is H. In some embodiments, R⁴ is D. In some embodiments, R⁴ is halogen. In some embodiments, R⁴ is Cl. In some embodiments, R⁴ is Br.

As defined generally above, R⁵ is H, D, or halogen.

In some embodiments, R⁵ is H. In some embodiments, R⁵ is D. In some embodiments, R⁵ is halogen. In some embodiments, R⁵ is Cl. In some embodiments, R⁵ is Br.

As defined generally above, R^{6a} is C₁₋₄ aliphatic optionally substituted with 1, 2, or 3 deuterium or halogen atoms.

In some embodiments, R^{6a} is C₁₋₄ aliphatic substituted with 1, 2, or 3 deuterium or halogen atoms. In some embodiments, R^{6a} is C₁₋₄ aliphatic. In some embodiments, R^{6a} is C₁₋₄ alkyl. In some embodiments, R^{6a} is methyl, ethyl, n-propyl, or isopropyl. In some embodiments, R^{6a} is methyl.

As defined generally above, R^{6b} is C₁₋₄ aliphatic optionally substituted with 1, 2, or 3 deuterium or halogen atoms.

In some embodiments, R^{6b} is C₁₋₄ aliphatic substituted with 1, 2, or 3 deuterium or halogen atoms. In some embodiments, R^{6b} is C₁₋₄ aliphatic. In some embodiments, R^{6b} is C₁₋₄ alkyl. In some embodiments, R^{6b} is C₁₋₄ alkyl optionally substituted with 1, 2, or 3 fluorine atoms. In some embodiments, R^{6b} is methyl, ethyl, n-propyl, or isopropyl. In some embodiments, R^{6b} is methyl.

In some embodiments, R^{6a} and R^{6b} are methyl or ethyl. In some embodiments, R^{6a} and R^{6b} are methyl. In some embodiments, R^{6a} and R^{6b} are —CD₃.

In some embodiments, the compound is of Formula II-a:

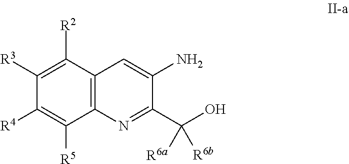

or a pharmaceutically acceptable salt thereof, wherein:
each of R², R³, R⁴, R⁵, R^{6a}, and R^{6b} is as defined as provided above and described in embodiments herein, both singly and in combination.

In some embodiments, the compound is of Formula II-b:

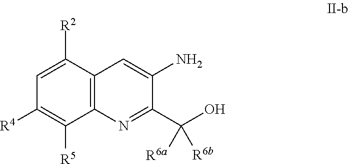

or a pharmaceutically acceptable salt thereof, wherein:
each of R², R⁴, R⁵, R^{6a}, and R^{6b} is as defined as provided above and described in embodiments herein, both singly and in combination.

In some embodiments, the compound is of any one of Formulae II-c, II-d, II-e, or II-f:

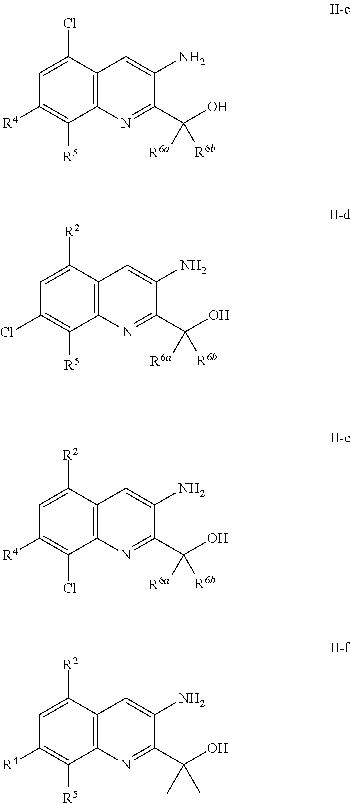

or a pharmaceutically acceptable salt thereof, wherein:
each of R², R⁴, R⁵, R^{6a}, and R^{6b} is as defined as provided above and described in embodiments herein, both singly and in combination.

In some embodiments, the compound is of Formula II-g:

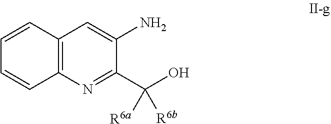

or a pharmaceutically acceptable salt thereof, wherein:
each of R^{6a} and R^{6b} is as defined as provided above and described in embodiments herein, both singly and in combination.

In some embodiments, a method of treatment described herein uses a compound selected from one depicted in Table 1, below, or a pharmaceutically acceptable salt thereof.

TABLE 1
Representative Compounds

| | |
|---|---|
| 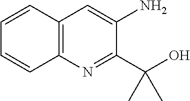 | I-1 (Compound 1) |
| 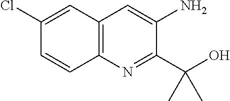 [SMILES: CC(C)(O)c1nc2ccc(Cl)cc2cc1N] | I-2 |
| 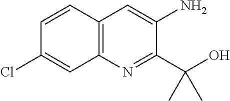 [SMILES: CC(C)(O)c1nc2cc(Cl)ccc2cc1N] | I-3 |
| 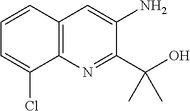 [SMILES: CC(C)(O)c1nc2c(Cl)cccc2cc1N] | I-4 |
| 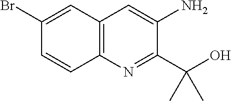 [SMILES: CC(C)(O)c1nc2ccc(Br)cc2cc1N] | 1-5 |
| 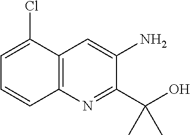 [SMILES: CC(C)(O)c1nc2cccc(Cl)c2cc1N] | I-6 |
| 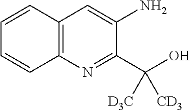 [SMILES: [2H]C([2H])([2H])C(O)(c1nc2ccccc2cc1N)C([2H])([2H])[2H]] | I-7 |
| 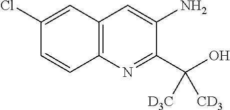 [SMILES: [2H]C([2H])([2H])C(O)(c1nc2ccc(Cl)cc2cc1N)C([2H])([2H])[2H]] | I-8 |
| 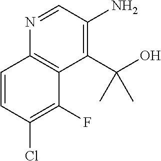 [SMILES: CC(C)(O)c1c(N)cnc2ccc(Cl)c(F)c12] | I-9 |
| 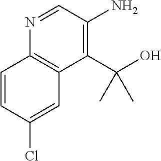 [SMILES: CC(C)(O)c1c(N)cnc2ccc(Cl)cc12] | I-10 |
| 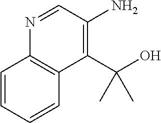 | I-11 |
| 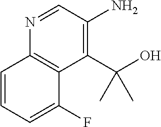 [SMILES: CC(C)(O)c1c(N)cnc2cccc(F)c12] | I-12 |
| 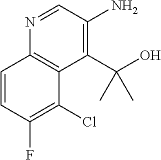 [SMILES: CC(C)(O)c1c(N)cnc2ccc(F)c(Cl)c12] | I-13 |
| 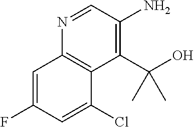 [SMILES: CC(C)(O)c1c(N)cnc2cc(F)cc(Cl)c12] | I-14 |
| 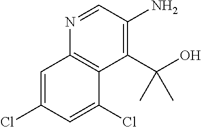 [SMILES: CC(C)(O)c1c(N)cnc2cc(Cl)cc(Cl)c12] | I-15 |
| 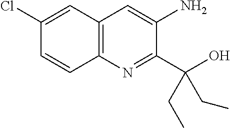 [SMILES: CCC(O)(CC)c1nc2ccc(Cl)cc2cc1N] | I-16 |
| 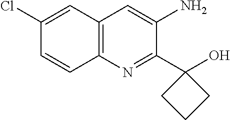 [SMILES: Nc1cc2cc(Cl)ccc2nc1C1(O)CCC1] | I-17 |
| 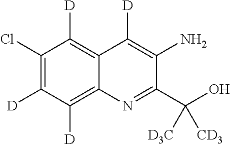 [SMILES: [2H]c1c(Cl)c([2H])c2c([2H])c(N)c(C(O)(C([2H])([2H])[2H])C([2H])([2H])[2H])nc2c1[2H]] | X-1 |
| 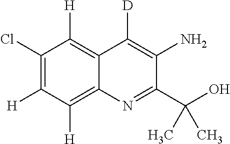 [SMILES: [2H]c1c(N)c(C(C)(C)O)nc2ccc(Cl)cc12] | X-2 |
| 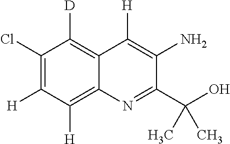 [SMILES: [2H]c1c(Cl)ccc2nc(C(C)(C)O)c(N)cc12] | X-3 |
| 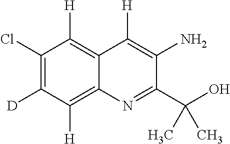 [SMILES: [2H]c1cc2nc(C(C)(C)O)c(N)cc2cc1Cl] | X-4 |
| 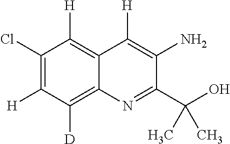 [SMILES: [2H]c1cc(Cl)cc2cc(N)c(C(C)(C)O)nc12] | X-5 |
| 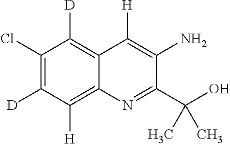 [SMILES: [2H]c1cc2nc(C(C)(C)O)c(N)cc2c([2H])c1Cl] | X-6 |
| 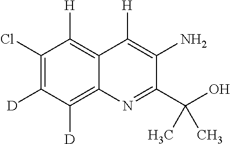 [SMILES: [2H]c1c(Cl)cc2cc(N)c(C(C)(C)O)nc2c1[2H]] | X-7 |
| 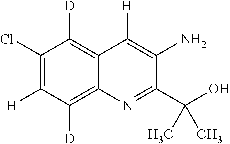 [SMILES: [2H]c1c(Cl)cc([2H])c2nc(C(C)(C)O)c(N)cc12] | X-8 |
| 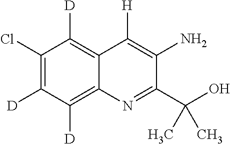 [SMILES: [2H]c1c(Cl)c([2H])c2cc(N)c(C(C)(C)O)nc2c1[2H]] | X-9 |
| 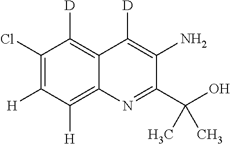 [SMILES: [2H]c1c(Cl)ccc2nc(C(C)(C)O)c(N)c([2H])c12] | X-10 |
| 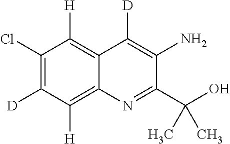 [SMILES: [2H]c1cc2nc(C(C)(C)O)c(N)c([2H])c2cc1Cl] | X-11 |
| 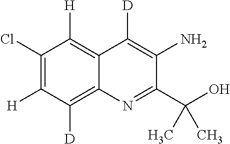 [SMILES: [2H]c1c(N)c(C(C)(C)O)nc2c([2H])cc(Cl)cc12] | X-12 |
| 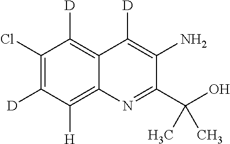 [SMILES: [2H]c1cc2nc(C(C)(C)O)c(N)c([2H])c2c([2H])c1Cl] | X-13 |
| 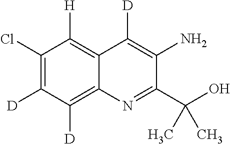 [SMILES: [2H]c1c(Cl)cc2c([2H])c(N)c(C(C)(C)O)nc2c1[2H]] | X-14 |
| 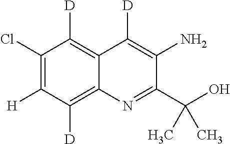 [SMILES: [2H]c1cc(Cl)c([2H])c2c([2H])c(N)c(C(C)(C)O)nc12] | X-15 |
| 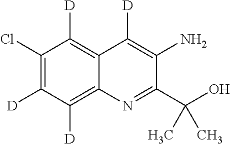 [SMILES: [2H]c1c(Cl)c([2H])c2c([2H])c(N)c(C(C)(C)O)nc2c1[2H]] | X-16 |
| 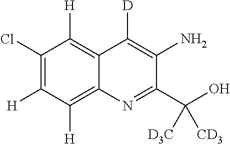 [SMILES: [2H]c1c(N)c(C(O)(C([2H])([2H])[2H])C([2H])([2H])[2H])nc2ccc(Cl)cc12] | X-17 |
| 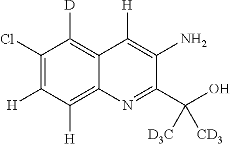 [SMILES: [2H]c1c(Cl)ccc2nc(C(O)(C([2H])([2H])[2H])C([2H])([2H])[2H])c(N)cc12] | X-18 |
| 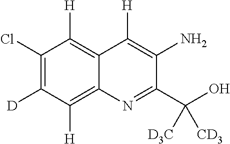 [SMILES: [2H]c1cc2nc(C(O)(C([2H])([2H])[2H])C([2H])([2H])[2H])c(N)cc2cc1Cl] | X-19 |
| 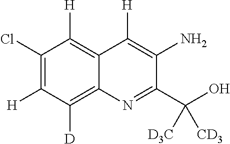 [SMILES: [2H]c1cc(Cl)cc2cc(N)c(C(O)(C([2H])([2H])[2H])C([2H])([2H])[2H])nc12] | X-20 |
| 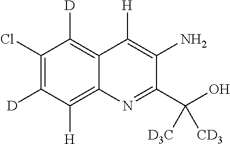 [SMILES: [2H]c1cc2nc(C(O)(C([2H])([2H])[2H])C([2H])([2H])[2H])c(N)cc2c([2H])c1Cl] | X-21 |
| 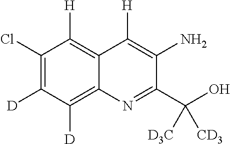 [SMILES: [2H]c1c(Cl)cc2cc(N)c(C(O)(C([2H])([2H])[2H])C([2H])([2H])[2H])nc2c1[2H]] | X-22 |
| 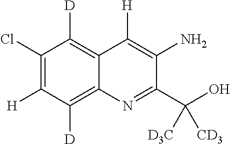 [SMILES: [2H]c1c(Cl)cc([2H])c2nc(C(O)(C([2H])([2H])[2H])C([2H])([2H])[2H])c(N)cc12] | X-23 |
| 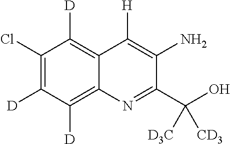 [SMILES: [2H]c1c(Cl)c([2H])c2cc(N)c(C(O)(C([2H])([2H])[2H])C([2H])([2H])[2H])nc2c1[2H]] | X-24 |
| 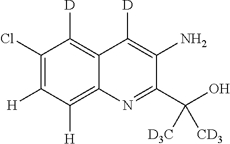 [SMILES: [2H]c1c(Cl)ccc2nc(C(O)(C([2H])([2H])[2H])C([2H])([2H])[2H])c(N)c([2H])c12] | X-25 |
| 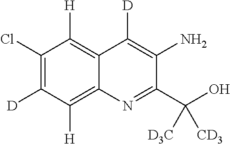 [SMILES: [2H]c1cc2nc(C(O)(C([2H])([2H])[2H])C([2H])([2H])[2H])c(N)c([2H])c2cc1Cl] | X-26 |
| 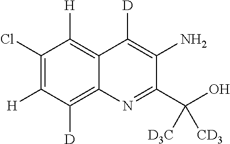 [SMILES: [2H]c1c(N)c(C(O)(C([2H])([2H])[2H])C([2H])([2H])[2H])nc2c([2H])cc(Cl)cc12] | X-27 |
| 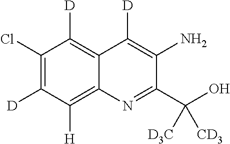 [SMILES: [2H]c1cc2nc(C(O)(C([2H])([2H])[2H])C([2H])([2H])[2H])c(N)c([2H])c2c([2H])c1Cl] | X-28 |
| 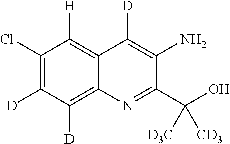 [SMILES: [2H]c1c(Cl)cc2c([2H])c(N)c(C(O)(C([2H])([2H])[2H])C([2H])([2H])[2H])nc2c1[2H]] | X-29 |
| 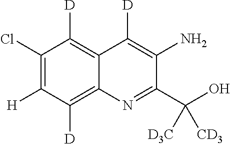 [SMILES: [2H]c1cc(Cl)c([2H])c2c([2H])c(N)c(C(O)(C([2H])([2H])[2H])C([2H])([2H])[2H])nc12] | X-30 |

In some embodiments, the present disclosure provides a compound or pharmaceutical composition comprising a compound depicted in Table 1, above, or a pharmaceutically acceptable salt thereof, and one or more pharmaceutically acceptable excipients or carriers.

In some embodiments, the present invention provides a unit dosage form comprising a disclosed compound or pharmaceutical composition.

In some embodiments, the unit dosage form comprises a spray-dried pharmaceutical composition comprising compound I-1, or a pharmaceutically acceptable salt thereof. In some embodiments, the unit dosage form is in the form of an oral-powder-for-constitution (OPC) formulation.

In some embodiments, the unit dosage form comprises about 1 mg to about 2000 mg of a disclosed quinoline compound, or a pharmaceutically acceptable salt thereof.

In some embodiments, the unit dosage form comprises about 1 mg to about 2000 mg I-1, or a pharmaceutically acceptable salt thereof.

In some embodiments, the unit dosage form comprises about 1 mg to about 1000 mg I-1, or a pharmaceutically acceptable salt thereof.

In some embodiments, the unit dosage form comprises about 1 mg to about 800 mg, 5 mg to about 500 mg, 10 mg to about 600 mg, or about 10 mg to about 350 mg I-1, or a pharmaceutically acceptable salt thereof.

In some embodiments, the unit dosage form comprises about 1 mg, about 5 mg, about 10 mg, about 30 mg, about 40 mg, about 350 mg, or about 450 mg of compound I-1, or a pharmaceutically acceptable salt thereof.

In some embodiments, the unit dosage form comprises about 10 mg, about 25 mg, about 50 mg, about 75 mg, about 100 mg, about 150 mg, about 200 mg, about 250 mg, about 300 mg, about 400 mg, about 500 mg, about 750 mg, or about 1000 mg of compound I-1, or a pharmaceutically acceptable salt thereof.

In some embodiments, the unit dosage form is suitable for systemic administration.

In some embodiments, the unit dosage form is suitable for parenteral or oral administration.

In some embodiments, the unit dosage form is suitable for oral administration.

In some embodiments, the present invention provides a unit dosage form comprising a pharmaceutical composition, in liquid form, comprising about 1 mg to about 2000 mg I-1, or a pharmaceutically acceptable salt thereof, and water.

In some embodiments, the unit dosage form is a capsule or tablet.

In some embodiments, the one or more pharmaceutically acceptable excipients or carriers are selected from one or more of Eudragit L100, microcrystalline cellulose, lactose monohydrate, croscarmellose sodium, crospovidone (Kollidon CL), a vinylpyrrolidone-vinyl acetate copolymer such as Kollidon VA64, sodium lauryl sulfate, and magnesium stearate.

In one aspect, the present disclosure provides a pharmaceutical composition comprising:
a) compound I-1:

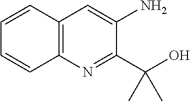

or a pharmaceutically acceptable salt thereof;
b) one or more fillers;
c) one or more binders;
d) one or more disintegrants;
e) one or more glidants;
f) one or more lubricants;
g) optionally, one or more surfactants; and
h) optionally, one or more effervescent components.

In some embodiments, compound I-1 or a pharmaceutically acceptable salt thereof is present in the pharmaceutical composition as a spray-dried solid (e.g., obtained from a spray drying dispersion (SDD)).

In some embodiments, the pharmaceutical composition exhibits pharmacokinetics results as described herein.

In some embodiments, the spray-dried solid comprises a concentration-enhancing polymer.

Exemplary concentration-enhancing polymers include those described in U.S. Pat. Nos. 7,780,988 and 10,004,719, each of which is hereby incorporated by reference. In some embodiments, the spray-dried solid is prepared substantially as described in U.S. Pat. No. 7,780,988 or U.S. Pat. No. 10,004,719, each of which is hereby incorporated by reference.

In some embodiments, the concentration-enhancing polymer is selected from selected from the group consisting of ionizable cellulosic polymers, non-ionizable cellulosic polymers, and ionizable non-cellulosic polymers, and blends thereof.

In some embodiments, the concentration-enhancing polymer is selected from the group consisting of hydroxypropyl methyl cellulose, hydroxypropyl cellulose, carboxymethyl ethyl cellulose, hydroxypropyl methyl cellulose acetate succinate, hydroxypropyl methyl cellulose phthalate, cellulose acetate phthalate, and cellulose acetate trimellitate, and blends thereof.

In some embodiments, the spray-dried solid comprises an anionic copolymer such as one based on methacrylic acid and methyl methacrylate. In some embodiments, the anionic copolymer is a Eudragit polymer. In some embodiments, the anionic copolymer is Eudragit L100.

In some embodiments, the pharmaceutical composition exhibits a PK result, after oral administration to a human subject, as described herein.

In some embodiments, the one or more fillers are selected from ammonium aliginate, calcium carbonate, calcium lactate, calcium phosphate, calcium silicate, calcium sulfate, cellulose acetate, compressible sugar (e.g., lactose, glucose, and sucrose), corn starch, dextrates, erythritol, ethyl cellulose, glyceryl palmitostearate, isomalt, kaolin, magnesium carbonate, magnesium oxide, maltodextrin, medium-chain triglycerides, microcrystalline cellulose, pre-gelatinized starch, polydextrose, polymethacrylates, silicic acid, simethicone, sodium alginate, sodium chloride, mannitol, sorbitol, starch, sugar spheres, sulfobutylether p-cyclodextrin, talc, tragacanth, trehalose, and xylitol, or a combination thereof.

In some embodiments, the filler is microcrystalline cellulose. In some embodiments, the filler is lactose. In some embodiments, the filler is starch. In some embodiments, the filler is a combination of starch and lactose.

In some embodiments, the filler is a combination of lactose, microcrystalline cellulose, and mannitol.

In some embodiments, the one or more binders are selected from acacia gum, agar, alginic acid, calcium carbonate, calcium lactate, carbomers (e.g., acrylic acid polymer, carboxy polymethylene, polyacrylic acid, carboxyvinyl polymer), carboxymethylcellulose sodium, carboxycellulose, carrageenan, cellulose acetate phthalate, ceratonia, chitosan, copovidone, corn starch, cottonseed oil, dextrates, dextrin, dextrose, ethylcellulose, gelatin, glyceryl behenate, guar gum, hydrogenated vegetable oil type I, hydroxyethylcellulose, hydroxyethylmethyl cellulose, hydroxypropyl cellulose, hydroxypropyl methylcellulose, hypromellose, inulin, lactose, magnesium aluminum silicate, maltodextrin, maltose, methylcellulose, microcrystalline cellulose, pectin, poloxamer, polycarbohil, polydextrose, polyethylene oxide, polymetharylates, polyvinylpyrrolidone, pre-gelatinized starch, povidone, sodium alginate, starch, stearic acid, sucrose, tricaprylin, vitamin E polyethylene glycol succinate, and zein.

In some embodiments, the binder is a vinylpyrrolidone-vinyl acetate copolymer. In some embodiments, the binder is Kollidon VA64.

In some embodiments, the one or more disintegrants are selected from agar, bentonite, celluloses (e.g., methylcellulose and carboxymethylcellulose), wood products, natural sponge, cation-exchange resins, alginic acid, gums (e.g., guar gum and Veegum HV), citrus pulp, cross-linked celluloses (e.g., croscarmellose), cross-linked polymers (e.g., crospovidone), cross-linked starches, calcium carbonate, microcrystalline cellulose (e.g., sodium starch glycolate), polacrilin potassium, starches (e.g., corn starch, potato starch, tapioca starch, and pre-gelatinized starch), clays, and aligns; and mixtures thereof.

In some embodiments, the one or more glidants are selected from colloidal silicon dioxide, CAB-O-SIL™ (Cabot Co. of Boston, MA), fumed silica (Aerosil), and asbestos-free talc.

In some embodiments, the one or more glidants are selected from colloidal silicon dioxide and fumed silica.

In some embodiments, the one or more lubricants are selected from calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. In some embodiments, the one or more lubricants are selected from a syloid silica gel (AEROSIL200), a coagulated aerosol of synthetic silica (marketed by Degussa Co. of Plano, Tex.), CAB-O-SIL™, and mixtures thereof.

In some embodiments, the one or more surfactants are selected from polyoxyethylene (20) sorbitan monolaurate (e.g., Tween-20), polyoxyethylene (20) sorbitan monooleate (e.g., Tween-80), sodium laurel sulfate, and sodium dodecyl sulfate.

In some embodiments, the surfactant comprises sodium lauryl sulfate.

In some embodiments, the one or more effervescent components are selected from a carbon dioxide-releasing component, such as a bicarbonate-containing component. In some embodiments, the effervescent component comprises sodium bicarbonate, such as a compressed mixture of sodium bicarbonate and an organic acid. In some embodiments, the organic acid is citric acid or tartaric acid.

In some embodiments, the pharmaceutical composition optionally comprises an antioxidant or chelating agent. In some embodiments, the antioxidant is ascorbyl palmitate, butylated hydroxyanisole, butylated hydroxytoluene (BHT), calcium stearate, citric acid, sodium thiosulfate, sodium metabisulfite, vitamin E, or 3,4-dihydroxybenzoic acid. In some embodiments, the chelating agent is EDTA (ethylenediamine tetraacetic acid) or disodium EDTA.

In some embodiments, the pharmaceutical composition optionally comprises one or more diluents. In some embodiments, the pharmaceutical composition comprises one or more diluents selected from dicalcium phosphate, calcium sulfate, lactose, cellulose, kaolin, mannitol, sodium chloride, dry starch, microcrystalline cellulose (e.g., AVICEL), microfine cellulose, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT), potassium chloride, sodium chloride, sorbitol, and talc; or ammonium alginate, calcium carbonate, calcium phosphate, calcium sulfate, cellulose acetate, compressible sugar, confectioner's sugar, dextrates, dextrin, dextrose, erythritol, ethylcellulose, fructose, fumaric acid, glyceryl palmitostearate, isomalt, kaolin, lacitol, lactose, mannitol, magnesium carbonate, magnesium oxide, maltodextrin, maltose, medium-chain triglycerides, microcrystalline cellulose, microcrystalline silicified cellulose, powered cellulose, polydextrose, polymethylacrylates, simethicone, sodium alginate, sodium chloride, sorbitol, starch, pregelatinized starch, sucrose, sulfobutylether-o-cyclodextrin, talc, tragacanth, trehalose, and xylitol.

In some embodiments, the pharmaceutical composition optionally further comprises one or more additional binders, fillers, diluents, disintegrants, wetting agents, lubricants, glidants, coloring agents, dye-migration inhibitors, sweetening agents, flavoring agents, emulsifying agents, suspending and dispersing agents, preservatives, antioxidants, chelating agents, solvents, non-aqueous liquids, organic acids, and sources of carbon dioxide.

In one aspect, the present disclosure provides a pharmaceutical composition comprising:
a) compound I-1:

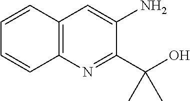

or a pharmaceutically acceptable salt thereof;
b) one or more fillers comprising at least one of microcrystalline cellulose, mannitol, and lactose monohydrate;
c) one or more binders comprising at least Kollidon VA64;
d) one or more disintegrants comprising at least one of croscarmellose sodium and crospovidone;
e) one or more glidants comprising fumed silica;
f) one or more lubricants comprising magnesium stearate;
g) optionally, one or more surfactants comprising lauryl sulfate; and
h) optionally, one or more effervescent components.

In some embodiments, compound I-1 or a pharmaceutically acceptable salt thereof is present in the pharmaceutical composition as a spray-dried solid (e.g., obtained from a spray drying dispersion (SDD)). In some embodiments, the spray-dried solid comprises an anionic copolymer such as one based on methacrylic acid and methyl methacrylate. In some embodiments, the anionic copolymer is a Eudragit polymer. In some embodiments, the anionic copolymer is Eudragit L 100.

In some embodiments, the unit dosage form exhibits pharmacokinetics results as described herein.

In one aspect, the present disclosure provides a pharmaceutical composition comprising:
a) compound I-1:

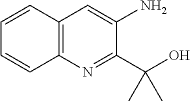

or a pharmaceutically acceptable salt thereof;
b) microcrystalline cellulose, mannitol, and lactose monohydrate;
c) Kollidon VA64;
d) croscarmellose sodium and crospovidone;
e) fumed silica;
f) magnesium stearate;
g) optionally, lauryl sulfate; and
h) optionally, one or more effervescent components.

In some embodiments, the lauryl sulfate is present. In some embodiments, the effervescent component is present and is a mixture of sodium bicarbonate and citric acid. In some embodiments, the effervescent component is Effer-soda and citric acid.

In some embodiments, compound I-1 or a pharmaceutically acceptable salt thereof is present in the pharmaceutical composition as a spray-dried solid (e.g., obtained from a spray drying dispersion (SDD)). In some embodiments, the spray-dried solid comprises an anionic copolymer such as one based on methacrylic acid and methyl methacrylate. In some embodiments, the anionic copolymer is a Eudragit polymer. In some embodiments, the anionic copolymer is Eudragit L 100.

In some embodiments, the unit dosage form exhibits pharmacokinetics results as described herein.

In one aspect, the present disclosure provides a pharmaceutical composition comprising:
a) a spray-dried solid comprising a mixture of Eudragit L 100 and compound I-1:

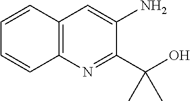

or a pharmaceutically acceptable salt thereof;
b) microcrystalline cellulose, mannitol, and lactose monohydrate;
c) Kollidon VA64;
d) croscarmellose sodium and crospovidone;
e) fumed silica;
f) magnesium stearate;
g) lauryl sulfate; and
h) optionally, one or more effervescent components.

In some embodiments, the effervescent component is not present. In some embodiments, the effervescent component is present and is a mixture of sodium bicarbonate, sodium carbonate, and citric acid.

In some embodiments, the pharmaceutical composition comprises about 5% to about 75% w/w of active ingredient. In some embodiments, the pharmaceutical composition comprises about 15% to about 60% w/w of active ingredient. In some embodiments, the pharmaceutical composition comprises about 20% to about 40% w/w of active ingredient. In some embodiments, the pharmaceutical composition comprises about 25% to about 37% w/w of active ingredient.

In some embodiments, the pharmaceutical composition comprises about 5%, 10%, 15%, 20%, 25%, 30%, 35%, 40%, 45%, 50%, 55%, 60%, 65%, 70%, 75%, 80%, 85%, 90%, or 95% w/w of active ingredient.

In some embodiments, the pharmaceutical composition comprises about 31% w/w of active ingredient.

In some embodiments, the active ingredient is compound I-1 or a pharmaceutically acceptable salt thereof.

In some embodiments, I-1 or a pharmaceutically acceptable salt thereof is provided as a spray-dried solid mixed with Eudragit L100. In some embodiments, about 0.9 mg to about 3.6 mg of Eudragit L100 is mixed with each 1 mg of I-1 or pharmaceutically acceptable salt thereof. In some embodiments, about 1.0 mg to about 3.0 mg of Eudragit L 100 is mixed with each 1 mg of I-1 or pharmaceutically acceptable salt thereof. In some embodiments, about 1.5 mg to about 2.2 mg of Eudragit L100 is mixed with each 1 mg of I-1 or pharmaceutically acceptable salt thereof. In some embodiments, about 1.8 mg of Eudragit L100 is mixed with each 1 mg of I-1 or pharmaceutically acceptable salt thereof.

In some embodiments, the pharmaceutical composition comprises about 16% to 40% w/w I-1 or pharmaceutically acceptable salt thereof and about 29.3% to about 80% w/w Eudragit L 100.

In some embodiments, the unit dosage form exhibits pharmacokinetics results as described herein.

In some embodiments, the pharmaceutical composition comprises about 1% to about 20% w/w of a filler described herein.

In some embodiments, the pharmaceutical composition comprises about 1% to about 20% w/w of a disintegrant described herein.

In some embodiments, the pharmaceutical composition comprises about 2% to about 18% w/w of a binder described herein. In some embodiments, the pharmaceutical composition comprises about 4% to about 16% w/w of a binder described herein. In some embodiments, the pharmaceutical composition comprises about 5% to about 10% w/w of a binder described herein. In some embodiments, the pharmaceutical composition comprises about 7% w/w of a binder described herein.

In some embodiments, the pharmaceutical composition comprises about 0.05% to about 2.0% w/w of a lubricant described herein. In some embodiments, the pharmaceutical composition comprises about 0.1% to about 1.0% w/w of a lubricant described herein. In some embodiments, the pharmaceutical composition comprises about 0.25% to about 0.75% w/w of a lubricant described herein. In some embodiments, the pharmaceutical composition comprises about 0.5% w/w of a lubricant described herein.

In some embodiments, the pharmaceutical composition comprises about 0.05% to about 2.0% w/w of a glidant described herein. In some embodiments, the pharmaceutical composition comprises about 0.1% to about 1.0% w/w of a glidant described herein. In some embodiments, the pharmaceutical composition comprises about 0.25% to about 0.75% w/w of a glidant described herein. In some embodiments, the pharmaceutical composition comprises about 0.5% w/w of a glidant described herein.

In some embodiments, the pharmaceutical composition comprises about 0.05% to about 2.0% w/w of a surfactant described herein. In some embodiments, the pharmaceutical composition comprises about 0.1% to about 1.0% w/w of a surfactant described herein. In some embodiments, the pharmaceutical composition comprises about 0.25% to about 0.75% w/w of a surfactant described herein. In some embodiments, the pharmaceutical composition comprises about 0.5% w/w of a surfactant described herein.

In some embodiments, the pharmaceutical composition comprises about 0.05% to about 2.0% w/w of an effervescent component described herein. In some embodiments, the pharmaceutical composition comprises about 0.1% to about 1.0% w/w of an effervescent component described herein. In some embodiments, the pharmaceutical composition comprises about 0.25% to about 0.75% w/w of an effervescent component described herein.

In some embodiments, the pharmaceutical composition comprises about 0.5% w/w of an effervescent component described herein.

In one aspect, the present disclosure provides a pharmaceutical composition comprising:

| | Quantity | % (w/w) |
|---|---|---|
| Component | (mg/unit) | of Total |
| Compound I-1 | 100 | 31.5% |
| Eudragit L100 | 185.7 | 58.5% |
| Pearlitol 200 SD (Mannitol) | 6.4 | 2.0% |
| Kollidon ® VA 64 Fine | 22.2 | 7.0% |
| Aerosil 200 Pharma (Fumed Silica) | 1.6 | 0.50% |
| Magnesium Stearate | 1.6 | 0.50% |
| Total | 317.5 | — |

In one aspect, the present disclosure provides a pharmaceutical composition comprising:

| No. | Component | Function |
|---|---|---|
| 1 | I-1 Crystalline: Eudragit L100 | active ingredient and anionic |
| | | copolymer excipient |
| 2 | Microcrystalline cellulose | Filler |
| | (Avicel PH102) | |
| 3 | Mannitol (Pearlitol 200 SD) | Filler |
| 4 | Lactose Monohydrate | Filler |
| 5 | Croscarmellose sodium | Disintegrant |
| | (Ac-Di-Sol) | |
| 6 | Crospovidone (Kollidon CL) | Disintegrant |
| 7 | Kollidon VA64 | Binder |
| 8 | Magnesium Stearate | Lubricant |
| 9 | Fumed Silica (Aerosil) | Glidant |
| 10 | Sodium Lauryl Sulphate | Surfactant |
| 11 | Citric Acid | Acidity regulator for |
| | | effervescent component |
| 12 | Effer-soda (surface modified | Effervescent component |
| | sodium bicarbonate/carbonate) | |
| 13 | Gelatin capsule | Encapsulation |
| 14 | Hydroxypropyl methylcellulose | Encapsulation |
| | (HPMC) capsule | |

In some embodiments, the present disclosure provides a unit dosage form comprising a pharmaceutical composition described herein.

In some embodiments, the unit dosage form is in the form of a capsule or tablet. In some embodiments, the capsule is a gelatin capsule. In some embodiments, the capsule is a hydroxypropyl methylcellulose (HPMC) capsule.

In some embodiments, the unit dosage form comprises about 1 mg to about 800 mg, 5 mg to about 500 mg, 10 mg to about 600 mg, or about 10 mg to about 350 mg I-1, or a pharmaceutically acceptable salt thereof.

In some embodiments, the unit dosage form comprises about 10 mg of compound I-1 or a pharmaceutically acceptable salt thereof. In some embodiments, the unit dosage form comprises about 100 mg of compound I-1 or a pharmaceutically acceptable salt thereof. In some embodiments, the unit dosage form comprises about 300 mg of compound I-1 or a pharmaceutically acceptable salt thereof.

In some embodiments, a unit dosage form is manufactured as non-sterile spray dried, granulated powder blend, filled in a capsule for oral delivery. In some embodiments, the unit dosage form is in a 10 mg or 100 mg unit dose capsule strength; 600 mg Powder-In-Bottle; or 250 mg or 300 mg unit dose tablet strength.

In some embodiments, the unit dosage form comprises the components listed in one of the tables below.

TABLE A
Quantitative Composition of 10 mg Capsule

Quantity

| Component | % w/w | mg/unit |
|---|---|---|
| Compound I-1 | 31.5% | 10 |
| Eudragit L100 | 58.5% | 18.57 |
| Pearlitol 200 SD (Mannitol) | 2.0% | 0.64 |
| Kollidon ® VA 64 Fine | 7.0% | 2.22 |
| Aerosil 200 Pharma (Fumed silica) | 0.5% | 0.16 |
| Magnesium Stearate, Vegetable Grade-Non | 0.5% | 0.16 |
| Bovine (Mg Stearate) | | |
| Total | 100.0 | 31.75 |

TABLE B
Quantitative Composition of 100 mg I-1 Capsule

Quantity

| Component | % w/w | mg/unit |
|---|---|---|
| Compound I-1 | 31.5% | 100 |
| Eudragit L100 | 58.5% | 185.7 |
| Pearlitol 200 SD (Mannitol) | 2.0% | 6.4 |
| Kollidon ® VA 64 Fine | 7.0% | 22.2 |
| Aerosil 200 Pharma (Fumed Silica) | 0.5% | 1.6 |
| Magnesium Stearate, Vegetable Grade-Non | 0.5% | 1.6 |
| Bovine (Mg Stearate) | | |
| Total | 100.0 | 317.46 |

TABLE C
Quantitative Composition of 600 mg I-1
Active Drug Product (Powder-in-Bottle)

Quantity

| Component | % w/w | mg/unit |
|---|---|---|
| Compound I-1 | 31.5% | 600 |
| Eudragit L100 | 58.5% | 1114.2 |
| Pearlitol 200 SD (Mannitol) | 2.0% | 38.4 |
| Kollidon ® VA 64 Fine | 7.0% | 132.2 |
| Aerosil 200 Pharma (Fumed silica) | 0.5% | 9.6 |
| Magnesium Stearate, Vegetable Grade-Non | 0.5% | 9.6 |
| Bovine (Mg Stearate) | | |
| Total | 100.0 | 1905 |

TABLE D
Quantitative Composition of I-1 Active
Drug Product - 300 mg Tablet

| | Quantity | |
|---|---|---|
| Component | % w/w | mg/unit |
| Compound I-1 | 30% | 300 |
| Eudragit L100 | 55% | 557.14 |
| Pearlitol 200 SD (Mannitol) | 2% | 20.3 |
| Kollidon ® VA 64 Fine | 7% | 71.0 |
| Aerosil 200 Pharma (Fumed silica) | 1% | 5.1 |
| Magnesium Stearate, Vegetable Grade-Non | 1% | 10.1 |
| Bovine (Mg Stearate) | | |
| Ac-Di-Sol ® SD-711 Croscarmellose Sodium | 5% | 50.4 |
| Total | 100.0 | 1014.04 |

TABLE E
Quantitative Composition of I-1 Active
Drug Product - 250 mg Tablet

| | Quantity | |
|---|---|---|
| Component | % w/w | mg/unit |
| Compound I-1 | 30% | 250 |
| Eudragit L100 | 55% | 464.3 |
| Pearlitol 200 SD (Mannitol) | 2% | 16.9 |
| Kollidon ® VA 64 Fine | 7% | 59.2 |
| Aerosil 200 Pharma (Fumed silica) | 1% | 4.2 |
| Magnesium Stearate, Vegetable Grade-Non | 1% | 8.4 |
| Bovine (Mg Stearate) | | |
| Ac-Di-Sol ® SD-711 Croscarmellose Sodium | 5% | 42 |
| Total | 100.0 | 845.0 |

In some embodiments, a unit dosage form of the present disclosure exhibits pharmacokinetics results as described herein.

In some embodiments, a unit dosage form described herein that comprises compound I-1, or a pharmaceutically acceptable salt thereof, upon oral administration to a human subject in a daily dose of 20 mg, 50 mg, 100 mg, 200 mg, 400 mg, 700 mg, or 1200 mg (or about such doses, as the case may be), provides a pharmacokinetic result shown in the table below:

TABLE 2
Exemplary PK Results

| | 20 mg | 50 mg | 100 mg | 200 mg | 400 mg | 700 mg | 1200 mg |
|---|---|---|---|---|---|---|---|
| Cₘₐₓ (ng/mL) | 26.4 | 56.7 | 140 | 459 | 869 | 2020 | 4220 |
| AUC_{inf} (ng*h/mL) | 50.0 | 156 | 433 | 1140 | 2730 | 6710 | 14500 |
| t_{1/2} (h) | NC | NC | 3.52 | 6.04 | 6.15 | 7.33 | 7.69 |
| Cₘₐₓ (ng/mL) | about | about | about | about | about | about | about |
| | 26.4 | 56.7 | 140 | 459 | 869 | 2020 | 4220 |
| AUC_{inf} (ng*h/mL) | about | about | about | about | about | about | about |
| | 50.0 | 156 | 433 | 1140 | 2730 | 6710 | 14500 |
| t_{1/2} (h) | | | about | about | about | about | about |
| | | | 3.52 | 6.04 | 6.15 | 7.33 | 7.69 |

In some embodiments, a unit dosage form described herein that comprises as an active ingredient compound I-1, or a pharmaceutically acceptable salt thereof, upon oral administration to a human subject in a dose of 50 mg BID, 150 mg BID, 350 mg BID, or 600 mg BID (or about such doses, as the case may be), provides a pharmacokinetic result shown in the table below:

TABLE 3
Exemplary PK Results

| | | Mean Cₘₐₓ | Mean AUC₀₋₁₂ | Mean t_{1/2} |
|---|---|---|---|---|
| Day | Dose | (ng/mL) | (h*ng/mL) | (h) |
| 1 | 50 mg BID | 70.2 | 148 | 3.49 |
| | 150 mg BID | 343 | 809 | 4.86 |
| | 350 mg BID | 1190 | 2950 | 5.67 |
| | 600 mg BID | 2230 | 6450 | 4.15 |
| 10 | 50 mg BID | 85.9 | 252 | 5.25 |
| | 150 mg BID | 320 | 1170 | 6.34 |
| | 350 mg BID | 1230 | 4340 | 6.83 |
| | 600 mg BID | 1700 | 7220 | 4.66 |
| 1 | about 50 mg BID | about 70.2 | about 148 | about 3.49 |
| | about 150 mg BID | about 343 | about 809 | about 4.86 |
| | about 350 mg BID | about 1190 | about 2950 | about 5.67 |
| | about 600 mg BID | about 2230 | about 6450 | about 4.15 |
| 10 | about 50 mg BID | about 85.9 | about 252 | about 5.25 |
| | about 150 mg BID | about 320 | about 1170 | about 6.34 |
| | about 350 mg BID | about 1230 | about 4340 | about 6.83 |
| | about 600 mg BID | about 1700 | about 7220 | about 4.66 |

In some embodiments, the human subject is a healthy human subject.

In some embodiments, the pharmacokinetic result described herein is obtained in a healthy human subject.

In some embodiments, a disclosed unit dosage form that comprises as an active ingredient compound I-1, or a pharmaceutically acceptable salt thereof, upon oral administration to a human subject in a dose of about 50 mg BID, 150 mg BID, 350 mg BID, or 600 mg BID, provides a pharmacokinetic result shown in the table above on day 1 of administration.

In some embodiments, a disclosed unit dosage form that comprises as an active ingredient compound I-1, or a pharmaceutically acceptable salt thereof, upon oral administration to a human subject in a dose of about 50 mg BID, 150 mg BID, 350 mg BID, or 600 mg BID, provides a pharmacokinetic result shown in the table above after day 1 but before day 10 of administration.

In some embodiments, a disclosed unit dosage form that comprises as an active ingredient compound I-1, or a pharmaceutically acceptable salt thereof, upon oral administration to a human subject in a dose of about 50 mg BID, 150 mg BID, 350 mg BID, or 600 mg BID, provides a pharmacokinetic result shown in the table above on day 10 of administration.

In some embodiments, a disclosed unit dosage form that comprises as an active ingredient compound I-1, or a pharmaceutically acceptable salt thereof, upon oral administration to a human subject in a dose of about 50 mg BID, 150 mg BID, 350 mg BID, or 600 mg BID, provides a pharmacokinetic result shown in the table above after day 10 of administration.

In some embodiments, the t_{1/2} in a human subject after administration of a disclosed unit dosage form is from about 3 to about 8 hours.

In some embodiments, the t_{1/2} in a human subject after administration of a disclosed unit dosage form is from about 3.5 to about 7.5 hours.

In some embodiments, the t_{1/2} in a human subject after administration of a disclosed unit dosage form is about 3.5, about 4.8, about 6.0, about 6.4, about 6.15, about 6.8, about 7.3, or about 7.7 hours.

In some embodiments, a disclosed unit dosage form comprising compound I-1, or a pharmaceutically acceptable salt thereof, provides an oral bioavailability of at least 70%.

In some embodiments, a disclosed unit dosage form comprising compound I-1, or a pharmaceutically acceptable salt thereof, provides an oral bioavailability of at least 75%.

In some embodiments, a disclosed unit dosage form comprising compound I-1, or a pharmaceutically acceptable salt thereof, provides an oral bioavailability of at least 80%.

In some embodiments, a disclosed unit dosage form comprising compound I-1, or a pharmaceutically acceptable salt thereof, provides an oral bioavailability of at least 80%, 85%, 90%, or 95%.

In some embodiments, the present disclosure provides a pharmaceutical formulation comprising a compound described herein, or a pharmaceutically acceptable salt thereof, for use in a disclosed method of treatment of a disease, disorder, or condition. As used herein, the terms “treatment,” “treat,” and “treating” refer to reversing, alleviating, delaying the onset of, or inhibiting the progress of a disease, disorder, or condition; or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). In some cases, treatment is continued after symptoms have resolved, for example to prevent, delay or lessen the severity of their recurrence.

In another aspect, the present invention provides a method for reducing levels of one or more toxic aldehydes in a subject, comprising administering to a subject in need thereof a pharmaceutical composition, as described herein.

In another aspect, the present invention provides a method for reducing levels of one or more toxic aldehydes in a biological sample, comprising contacting the biological sample with a pharmaceutical composition, as described herein. In some embodiments, the method is carried out in vitro.

In some embodiments, the dose of the compound or pharmaceutically acceptable salt thereof is about 10 mg to about 10,000 mg per day. In some embodiments, the dose of the compound or pharmaceutically acceptable salt thereof is about 10 mg to about 7500 mg per day. In some embodiments, the dose of the compound or pharmaceutically acceptable salt thereof is about 50 mg to about 3600 mg per day. In some embodiments, the dose of the compound or pharmaceutically acceptable salt thereof is about 150 mg to about 1200 mg per day. In some embodiments, the dose of the compound or pharmaceutically acceptable salt thereof is about 250 mg to about 2400 mg per day. In some embodiments, the dose of the compound or pharmaceutically acceptable salt thereof is about 600 mg to about 5000 mg per day. In some embodiments, the dose of the compound or pharmaceutically acceptable salt thereof is about 1000 mg to about 7500 mg per day. In some embodiments, the dose of the compound or pharmaceutically acceptable salt thereof is about 400 mg to about 1200 mg per day. In some embodiments, the dose of the compound or pharmaceutically acceptable salt thereof is about 500 mg to about 1000 mg per day. In some embodiments, the dose of the compound or pharmaceutically acceptable salt thereof is about 300 mg to about 1000 mg per day. In some embodiments, the dose of the compound or pharmaceutically acceptable salt thereof is about 400 mg to about 800 mg per day. In some embodiments, the dose of the compound or pharmaceutically acceptable salt thereof is about 500 mg to about 700 mg per day. In some embodiments, the dose of the compound or pharmaceutically acceptable salt thereof is about 600 mg to about 1200 mg per day. In some embodiments, the dose of the compound or pharmaceutically acceptable salt thereof is about 250 mg to about 2400 mg per day. In some embodiments, the dose of the compound or pharmaceutically acceptable salt thereof is about 600 mg to about 5000 mg per day. In some embodiments, the dose of the compound or pharmaceutically acceptable salt thereof is about 1000 mg to about 7500 mg per day.

In some embodiments, the compound or pharmaceutically acceptable salt thereof is administered once, twice, thrice, or four times per day. In some embodiments, the compound or pharmaceutically acceptable salt thereof is administered twice per day.

In some embodiments, the dose of the compound or pharmaceutically acceptable salt thereof is about 600 mg BID (i.e., twice per day); 1.2 g BID; or 2.4 g BID.

In some embodiments, the dose of the compound or pharmaceutically acceptable salt thereof is about 200 mg BID, 300 mg BID, 400 mg BID, 500 mg BID, 600 mg BID, 700 mg BID, or 800 mg BID.

In some embodiments, the dose of the compound or pharmaceutically acceptable salt thereof is about 300 mg BID.

In some embodiments, the dose of the compound or pharmaceutically acceptable salt thereof is administered systemically.

In some embodiments, the dose of the compound or pharmaceutically acceptable salt thereof is administered orally.

In some embodiments, the dose of the compound or pharmaceutically acceptable salt thereof is administered to the patient in a fasted state. In some embodiments, the patient has consumed no food for at least 2 hours prior to dosing and at least 1 hour after dosing.

In some embodiments, the compound is I-1 or a pharmaceutically acceptable salt thereof. In some embodiments, the compound is I-2 or a pharmaceutically acceptable salt thereof.

In some embodiments, the compound reduces systemic inflammation in the patient.

In some embodiments, the compound reduces plasma levels of a biomarker selected from IL-10, IL-6, IL-10, and tumor necrosis factor alpha. In some embodiments, the compound reduces plasma levels of a biomarker selected from a RASP. In some embodiments, the RASP is malondialdehyde (MDA) and/or 4-hydroxynonenal (4-HNE).

In some embodiments, the method further comprises a reduction in the level of a reactive aldehyde species (RASP) in the patient's blood, such as malondialdehyde (MDA) or 4-hydroxynonenal (HNE).

In some embodiments, the level of RASP is reduced by at least 30%, at least 40%, at least 50%. In some embodiments, the level of RASP is reduced by about 30% to 75%. In some embodiments, the level of RASP is reduced by about 20% to about 60%, or about 20% to about 50%, or about 20% to about 30%.

### 4. Pharmaceutical Compositions, Administration, and Dosages

The compounds and compositions, according to the methods of the present invention, are administered using any amount and any route of administration effective for treating or lessening the severity of a disease described above. The exact amount required will vary from subject to subject, depending on the species, age, and general condition of the subject, the severity of the infection, the particular agent, its mode of administration, and the like. Compounds of the invention are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracistemally, intrathecally, transdermally, transmucosally, ophthalmically, via inhalation, intravaginally, intraperitoneally, topically (as by powders, ointments, or drops), buccally, intranasally, as an oral or nasal spray, or the like, depending on the severity of the disease being treated.

A unit dosage form of the invention can be formulated for oral administration. Pharmaceutical compositions/formulations that are suitable for oral administration can be provided as discrete dosage forms, such as, but not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In some embodiments, such dosage forms contain predetermined amounts of active ingredients, and may be prepared by methods of pharmacy known to those skilled in the art. See generally, Remington's Pharmaceutical Sciences, 18^{th} ed., Mack Publishing, Easton Pa. (1990). As used herein, oral administration also includes buccal, lingual, and sublingual administration.

In some embodiments, the formulation further comprises one or more pharmaceutically acceptable excipients or carriers.

A person of ordinary skill would recognize that pharmaceutical formulation ingredients may serve multiple purposes within a formulation. Accordingly, a person of ordinary skill would recognize that certain formulation components may be classified according to multiple functions (e.g., a component may be both a filler and a binder).

In some embodiments, a unit dosage form provided herein are prepared by combining the active ingredients in an intimate admixture with one or more pharmaceutically acceptable excipients or carriers, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide, according to conventional pharmaceutical compounding techniques. Excipients or carriers can take a wide variety of forms depending on the form of preparation desired for administration. For example, excipients or carriers suitable for use in oral liquid or aerosol dosage forms include, but are not limited to, water, glycols, oils, alcohols, flavoring agents, preservatives, and coloring agents. Examples of excipients or carriers suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In some embodiments, the active ingredient, such as compound I-1 or a pharmaceutically acceptable salt thereof, is incorporated into the pharmaceutical composition as spray-dried powder or granules. The use of spray-drying to produce powders from fluid feed stocks is well known, with applications ranging from powdered milk to bulk chemicals and pharmaceuticals. See U.S. Pat. No. 4,187,617 and Mujumbar et al., 91 Drying, pages 56-73 (1991). The use of spray-drying to form solid amorphous dispersions of drugs and concentration-enhancing polymers is also known. See commonly owned European Patent Applications Nos. 0 901 786, 1 027 886, 1 027 887, 1 027 888, and commonly owned PCT Applications Nos. WO 00/168092 and WO 00/168055, each of which is hereby incorporated by reference. A typical spray-drying apparatus comprises a drying chamber, atomizing means for atomizing a solvent-containing liquid feed into the drying chamber, a source of heated drying gas directed into the drying chamber and dried product collection means for separating the dried product from the cooled drying gas and vaporized solvent stream following its exit from the drying chamber. Examples of such apparatus include Niro Models PSD-1, PSD-2 and PSD-4 (Niro A/S, Soeborg, Denmark).

The spray-dried powder or granules generally include the active compound in combination with a polymer such as a concentration-enhancing polymer. One class of polymers suitable for use with the present invention comprises non-ionizable (neutral) non-cellulosic polymers. Exemplary polymers include: vinyl polymers and copolymers having at least one substituent selected from the group consisting of hydroxyl, alkylacyloxy, and cyclicamido; polyvinyl alcohols that have at least a portion of their repeat units in the unhydrolyzed (vinyl acetate) form; polyvinyl alcohol polyvinyl acetate copolymers; polyvinyl pyrrolidone; and polyethylene polyvinyl alcohol copolymers; and polyoxyethylene-polyoxypropylene copolymers.

Exemplary neutral non-cellulosic polymers are comprised of vinyl copolymers of at least one hydrophilic, hydroxyl-containing repeat unit and at least one hydrophobic, alkyl- or aryl-containing repeat unit. Such neutral vinyl copolymers are termed “amphiphilic hydroxyl-functional vinyl copolymers.” Amphiphilic hydroxyl-functional vinyl copolymers are believed to provide high concentration enhancements due to the amphiphilicity of these copolymers which provide both sufficient hydrophobic groups to interact with the hydrophobic, low-solubility drugs and also sufficient hydrophilic groups to have sufficient aqueous solubility for good dissolution. The copolymeric structure of the amphiphilic hydroxyl-functional vinyl copolymers also allows their hydrophilicity and hydrophobicity to be adjusted to maximize performance with a specific low-solubility drug.

Another class of polymers suitable for use with the present invention comprises ionizable non-cellulosic polymers. Exemplary polymers include: carboxylic acid-functionalized vinyl polymers, such as the carboxylic acid functionalized polymethacrylates and carboxylic acid functionalized polyacrylates such as the EUDRAGIT™ series manufactured by Rohm Tech Inc., of Malden. Mass.; amine-functionalized polyacrylates and polymethacrylates; proteins such as gelatin and albumin; and carboxylic acid functionalized starches such as starch glycolate.

Non-cellulosic polymers that are amphiphilic are copolymers of a relatively hydrophilic and a relatively hydrophobic monomer. Examples include acrylate and methacrylate copolymers. Exemplary commercial grades of such copolymers include the EUDRAGIT™ series, which are copolymers of methacrylates and acrylates.

An additional class of polymers comprises ionizable and neutral (or non-ionizable) cellulosic polymers with at least one ester- and/or ether-linked substituent in which the polymer has a degree of substitution of at least 0.05 for each substituent. It should be noted that in the polymer nomenclature used herein, ether-linked substituents are recited prior to “cellulose” as the moiety attached to the ether group; for example, “ethylbenzoic acid cellulose” has ethoxybenzoic acid substituents. Analogously, ester-linked substituents are recited after “cellulose” as the carboxylate; for example, “cellulose phthalate” has one carboxylic acid of each phthalate moiety ester-linked to the polymer and the other carboxylic acid unreacted.

It should also be noted that a polymer name such as “cellulose acetate phthalate” (CAP) refers to any of the family of cellulosic polymers that have acetate and phthalate groups attached via ester linkages to a significant fraction of the cellulosic polymers hydroxyl groups. Generally, the degree of substitution of each substituent group can range from 0.05 to 2.9 as long as the other criteria of the polymer are met. “Degree of substitution” refers to the average number of the three hydroxyls per saccharide repeat unit on the cellulose chain that have been substituted. For example, if all of the hydroxyls on the cellulose chain have been phthalate-substituted, the phthalate degree of substitution is 3. Also included within each polymer family type are cellulosic polymers that have additional substituents added in relatively small amounts that do not substantially alter the performance of the polymer.

Amphiphilic cellulosics comprise polymers in which the parent cellulosic polymer has been substituted at any or all of the 3 hydroxyl groups present on each saccharide repeat unit with at least one relatively hydrophobic substituent. Hydrophobic substituents may be essentially any substituent that, if substituted to a high enough level or degree of substitution, can render the cellulosic polymer essentially aqueous-insoluble. Examples of hydrophobic substituent include ether-linked alkyl groups such as methyl, ethyl, propyl, butyl, etc.; or ester-linked alkyl groups such as acetate, propionate, butyrate, etc.; and ether- and/or ester-linked aryl groups such as phenyl, benzoate, or phenylate. Hydrophilic regions of the polymer can be either those portions that are relatively unsubstituted, since the unsubstituted hydroxyls are themselves relatively hydrophilic, or those regions that are substituted with hydrophilic substituents. Hydrophilic substituents include ether- or ester-linked nonionizable groups such as the hydroxy alkyl substituents hydroxyethyl, hydroxypropyl, and the alkyl ether groups such as ethoxyethoxy or methoxyethoxy. Particularly preferred hydrophilic substituents are those that are ether- or ester-linked ionizable groups such as carboxylic acids, thiocarboxylic acids, substituted phenoxy groups, amines, phosphates or sulfonates.

One class of cellulosic polymers comprises neutral polymers, meaning that the polymers are substantially non-ionizable in aqueous solution. Such polymers contain non-ionizable substituents, which may be either ether-linked or ester-linked. Exemplary ether-linked non-ionizable substituents include: alkyl groups, such as methyl, ethyl, propyl, butyl, etc.; hydroxy alkyl groups such as hydroxymethyl, hydroxyethyl, hydroxypropyl, etc.; and aryl groups such as phenyl. Exemplary ester-linked non-ionizable substituents include: alkyl groups, such as acetate, propionate, butyrate, etc.; and aryl groups such as phenylate. However, when aryl groups are included, the polymer may need to include a sufficient amount of a hydrophilic substituent so that the polymer has at least some water solubility at any physiologically relevant pH of from 1 to 8.

Exemplary nonionizable cellulosic polymers that may be used as the polymer include: hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

An exemplary class of neutral cellulosic polymers are those that are amphiphilic. Exemplary polymers include hydroxypropyl methyl cellulose and hydroxypropyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer.

A particular class of cellulosic polymers comprises polymers that are at least partially ionizable at physiologically relevant pH and include at least one ionizable substituent, which may be either ether-linked or ester-linked. Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid such as ethoxynicotinic acid, and the various isomers of picolinic acid such as ethoxypicolinic acid, etc.; thiocarboxylic acids, such as thioacetic acid; substituted phenoxy groups, such as hydroxyphenoxy, etc.; amines, such as aminoethoxy, diethylaminoethoxy, trimethylaminoethoxy, etc.; phosphates, such as phosphate ethoxy; and sulfonates, such as sulphonate ethoxy. Exemplary ester-linked ionizable substituents include: carboxylic acids, such as succinate, citrate, phthalate, terephthalate, isophthalate, trimellitate, and the various isomers of pyridinedicarboxylic acid, etc.; thiocarboxylic acids, such as thiosuccinate; substituted phenoxy groups, such as amino salicylic acid; amines, such as natural or synthetic amino acids, such as alanine or phenylalanine; phosphates, such as acetyl phosphate; and sulfonates, such as acetyl sulfonate. For aromatic-substituted polymers to also have the requisite aqueous solubility, it is also desirable that sufficient hydrophilic groups such as hydroxypropyl or carboxylic acid functional groups be attached to the polymer to render the polymer aqueous soluble at least at pH values where any ionizable groups are ionized. In some cases, the aromatic substituent may itself be ionizable, such as phthalate or trimellitate substituents.

Exemplary cellulosic polymers that are at least partially-ionized at physiologically relevant pHs include: hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, carboxymethyl ethyl cellulose, ethyl carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Exemplary cellulosic polymers that meet the definition of amphiphilic, having hydrophilic and hydrophobic regions include polymers such as cellulose acetate phthalate and cellulose acetate trimellitate where the cellulosic repeat units that have one or more acetate substituents are hydrophobic relative to those that have no acetate substituents or have one or more ionized phthalate or trimellitate substituents.

A further subset of cellulosic ionizable polymers are those that possess both a carboxylic acid functional aromatic substituent and an alkylate substituent and thus are amphiphilic. Exemplary polymers include cellulose acetate phthalate, methyl cellulose acetate phthalate, ethyl cellulose acetate phthalate, hydroxypropyl cellulose acetate phthalate, hydroxylpropyl methyl cellulose phthalate, hydroxypropyl methyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Another subset of cellulosic ionizable polymers are those that possess a non-aromatic carboxylate substituent. Exemplary polymers include hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, hydroxyethyl cellulose acetate succinate and carboxymethyl ethyl cellulose. Of these cellulosic polymers that are at least partially ionized at physiologically relevant pHs, the inventors have found the following to be most preferred: hydroxypropyl methyl cellulose acetate succinate, hydroxypropyl methyl cellulose phthalate, cellulose acetate phthalate, cellulose acetate trimellitate and carboxymethyl ethyl cellulose. The most preferred is hydroxypropyl methyl cellulose acetate succinate (HPMCAS).

Another class of polymers consists of neutralized acidic polymers. By “neutralized acidic polymer” is meant any acidic polymer for which a significant fraction of the “acidic moieties” or “acidic substituents” have been “neutralized”; that is, exist in their deprotonated form. By “neutralized acidic cellulosic polymers” is meant any cellulosic “acidic polymer” in which a significant fraction of the “acidic moieties” or “acidic substituents” have been “neutralized.” By “acidic polymer” is meant any polymer that possesses a significant number of acidic moieties. In general, a significant number of acidic moieties would be greater than or equal to about 0.1 milliequivalents of acidic moieties per gram of polymer. “Acidic moieties” include any functional groups that are sufficiently acidic that, in contact with or dissolved in water, can at least partially donate a hydrogen cation to water and thus increase the hydrogen-ion concentration. This definition includes any functional group or “substituent,” as it is termed when the functional group is covalently attached to a polymer that has a pKa of less than about 10. Exemplary classes of functional groups that are included in the above description include carboxylic acids, thiocarboxylic acids, phosphates, phenolic groups, and sulfonates. Such functional groups may make up the primary structure of the polymer such as for polyacrylic acid, but more generally are covalently attached to the backbone of the parent polymer and thus are termed “substituents.”

The amount of concentration-enhancing polymer relative to the amount of drug present in the spray-dried dispersions depends on the drug and concentration-enhancing polymer and may vary widely from a drug-to-polymer weight ratio of 0.01 to 5. However, in most cases, except when the drug dose is quite low, e.g., 25 mg or less, it is preferred that the drug-to-polymer ratio is greater than 0.05 and less than 2.5 and often the enhancement in drug concentration or relative bioavailability is observed at drug-to-polymer ratios of 1 or less or for some drugs even 0.2 or less. In cases where the drug dose is about 25 mg or less, the drug-to-polymer weight ratio may be significantly less than 0.05. In general, regardless of the dose, enhancements in drug concentration or relative bioavailability increase with decreasing drug-to-polymer weight ratio. However, due to the practical limits of keeping the total mass of a tablet, capsule or suspension low, it is often desirable to use a relatively high drug-to-polymer ratio as long as satisfactory results are obtained. The maximum drug:polymer ratio that yields satisfactory results varies from drug to drug and is best determined in the in vitro and/or in vivo dissolution tests described below.

A spray-dried solid as described herein can be a solid dispersion that contains a compound described herein and a pharmaceutically acceptable polymer. Certain compounds described herein generally have low aqueous solubility, and their absorption in vivo is dissolution-rate limited. A solid dispersion containing a compound can increase the compound solubility/dissolution, thereby improving the bioavailability of the compound.

The term “solid dispersion” herein refers to the dispersion of a pharmaceutically active ingredient, e.g., the compound described herein, in an inert polymer matrix at solid state. A solid dispersion can be prepared by methods well known in the art, e.g., spray-drying or hot-melt extrusion. The matrix can be either crystalline or amorphous. A solid dispersion contains a co-precipitate of a pharmaceutically active ingredient and one or more water-soluble polymers, in which the pharmaceutically active ingredient is dispersed uniformly within a polymer matrix formed from the polymers. The pharmaceutically active ingredient can be present in an amorphous state, a crystalline dispersed form, or a combination thereof. It can also be finely dispersed or dissolved as single molecules in the polymer matrix. The solid dispersion is typically prepared by a spray-drying method or a hot-melt extrusion method.

The method for preparing the solid dispersion includes steps of (i) mixing a compound described herein and a polymer in an organic solvent to provide a feeder solution and (ii) spray-drying the feeder solution through a nozzle as a fine spray into a chamber where the solvent is evaporated quickly to generate particles containing the compound and polymer. Following formation of a solid dispersion, the resulting spray-dried particle can undergo a secondary drying step to remove residual solvents. The secondary drying step can take place in a static dryer or an agitated dryer. Gas, humidified gas, vacuum can be applied to the secondary drying step and such application is useful in more rapidly removing residual solvents that remain in the spray-dried particle.

Any organic solvent that can easily dissolve or disperse the compound and the polymer described above can be used. Examples of the organic solvent include lower carbon-number alcohols, e.g., methanol, ethanol, propanol, and isopropanol; ketones, e.g., methylethyl ketone and butanone; and a combination thereof.

In some embodiments, the pharmaceutically acceptable excipients and carriers are selected from fillers, binders, diluents, disintegrants, glidants, and lubricants.

In some embodiments, the present invention provides a capsule or tablet which comprises a provided pharmaceutical composition in the form of a solid dosage form. In some embodiments, the present invention provides a capsule. In some embodiments, the present invention provides a tablet.

In certain embodiments, the dosage form is a tablet, wherein the tablet is manufactured using standard, art-recognized tablet processing procedures and equipment. In certain embodiments, the method for forming the tablets is direct compression of a powdered, crystalline and/or granular composition comprising a solid form provided herein, alone or in combination with one or more excipients or carriers, such as, for example, carriers, additives, polymers, or the like. In certain embodiments, as an alternative to direct compression, the tablets may be prepared using wet granulation or dry granulation processes. In certain embodiments, the tablets are molded rather than compressed, starting with a moist or otherwise tractable material. In certain embodiments, compression and granulation techniques are used.

In certain embodiments, the dosage form is a capsule, wherein the capsules may be manufactured using standard, art-recognized capsule processing procedures and equipments. In certain embodiments, soft gelatin capsules may be prepared in which the capsules contain a mixture comprising a solid form provided herein and vegetable oil or non-aqueous, water miscible materials, such as, for example, polyethylene glycol and the like. In certain embodiments, hard gelatin capsules may be prepared containing granules of solid forms provided herein in combination with a solid pulverulent carrier, such as, for example, lactose, saccharose, sorbitol, mannitol, potato starch, corn starch, amylopectin, cellulose derivatives, or gelatin. In certain embodiments, a hard gelatin capsule shell may be prepared from a capsule composition comprising gelatin and a small amount of plasticizer such as glycerol. In certain embodiments, as an alternative to gelatin, the capsule shell may be made of a carbohydrate material. In certain embodiments, the capsule composition may additionally include polymers, colorings, flavorings and opacifiers as required. In certain embodiments, the capsule comprises HPMC.

In some embodiments, the pharmaceutical composition comprises one or more fillers. In certain embodiments, the filler is selected from ammonium aliginate, calcium carbonate, calcium lactate, calcium phosphate, calcium silicate, calcium sulfate, cellulose acetate, compressible sugar (e.g., lactose, glucose, and sucrose), corn starch, dextrates, erythritol, ethyl cellulose, glyceryl palmitostearate, isomalt, kaolin, magnesium carbonate, magnesium oxide, maltodextrin, medium-chain triglycerides, microcrystalline cellulose, pre-gelatinized starch, polydextrose, polymethacrylates, silicic acid, simethicone, sodium alginate, sodium chloride, sorbitol, starch, sugar spheres, sulfobutylether β-cyclodextrin, talc, tragacanth, trehalose, and xylitol, or a combination thereof.

In some embodiments, the filler is selected from talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

In some embodiments, the filler is microcrystalline cellulose. In some embodiments, the filler is lactose. In some embodiments, the filler is starch. In some embodiments, the filler is a combination of starch and lactose. In some embodiments, the filler is a combination of lactose and microcrystalline cellulose. In some embodiments, the filler is a combination of two or three components recited above. In some embodiments, the filler comprises at least microcrystalline cellulose, lactose, and mannitol.

In certain embodiments, dosage forms provided herein comprise one or more diluents. Diluents may be used, e.g., to increase bulk so that a practical size tablet or capsule is ultimately provided. Suitable diluents include dicalcium phosphate, calcium sulfate, lactose, cellulose, kaolin, mannitol, sodium chloride, dry starch, microcrystalline cellulose (e.g., AVICEL), microfine cellulose, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT), potassium chloride, sodium chloride, sorbitol and talc, among others. Diluents also include, e.g., ammonium alginate, calcium carbonate, calcium phosphate, calcium sulfate, cellulose acetate, compressible sugar, confectioner's sugar, dextrates, dextrin, dextrose, erythritol, ethylcellulose, fructose, fumaric acid, glyceryl palmitostearate, isomalt, kaolin, lacitol, lactose, mannitol, magnesium carbonate, magnesium oxide, maltodextrin, maltose, medium-chain triglycerides, microcrystalline cellulose, microcrystalline silicified cellulose, powered cellulose, polydextrose, polymethylacrylates, simethicone, sodium alginate, sodium chloride, sorbitol, starch, pregelatinized starch, sucrose, sulfobutylether-o-cyclodextrin, talc, tragacanth, trehalose, and xylitol.

In some embodiments, the pharmaceutical composition comprises one or more binders. Binders may be used, e.g., to impart cohesive qualities to a tablet or a capsule, and thus ensure that the formulation remains intact after compression. In some embodiments, the binder is selected from acacia gum, agar, alginic acid, calcium carbonate, calcium lactate, carbomers (e.g., acrylic acid polymer, carboxy polymethylene, polyacrylic acid, carboxyvinyl polymer), carboxymethylcellulose sodium, carrageenan, cellulose acetate phthalate, ceratonia, chitosan, copovidone, corn starch, cottonseed oil, dextrates, dextrin, dextrose, ethylcellulose, gelatin, glyceryl behenate, guar gum, hydrogenated vegetable oil type 1, hydroxyethylcellulose, hydroxyethylmethyl cellulose, hydroxypropyl cellulose, hydroxypropyl methylcellulose, hypromellose, inulin, lactose, magnesium aluminum silicate, maltodextrin, maltose, methylcellulose, microcrystalline cellulose, pectin, poloxamer, polycarbohil, polydextrose, polyethylene oxide, polymetharylates, polyvinylpyrrolidone, pre-gelatinized starch, povidone, sodium alginate, starch, stearic acid, sucrose, tricaprylin, vitamin E polyethylene glycol succinate, and zein.

Suitable binders include, but are not limited to, starch (including potato starch, corn starch, and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone (PVP), cellulosic polymers (including hydroxypropyl cellulose (HPC), hydroxypropylmethylcellulose (HPMC), methyl cellulose, ethyl cellulose, hydroxyethyl cellulose (HEC), carboxymethyl cellulose and the like), veegum, carbomer (e.g., carbopol), sodium, dextrin, guar gum, hydrogenated vegetable oil, magnesium aluminum silicate, maltodextrin, polymethacrylates, povidone (e.g., KOLLIDON, PLASDONE), microcrystalline cellulose, among others. Binding agents also include, e.g., acacia, agar, alginic acid, carbomers, carrageenan, cellulose acetate phthalate, ceratonia, chitosan, confectioner's sugar, copovidone, dextrates, dextrin, dextrose, ethylcellulose, gelatin, glyceryl behenate, guar gum, hydroxyethyl cellulose, hydroxyethylmethyl cellulose, hydroxypropyl cellulose, hydroxypropyl starch, hypromellose, inulin, lactose, magnesium aluminum silicate, maltodextrin, maltose, methylcellulose, poloxamer, polycarbophil, polydextrose, polyethylene oxide, polymethylacrylates, povidone, sodium alginate, sodium carboxymethylcellulose, starch, pregelatinized starch, stearic acid, sucrose, and zein.

Suitable forms of microcrystalline cellulose include, but are not limited to, the materials sold as AVICEL-PH-101, AVICEL-PH-103 AVICEL RC-581, AVICEL-PH-105 (FMC Corporation, Marcus Hook, Pa.), and mixtures thereof. In some embodiment, a specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

In some embodiments, the pharmaceutical composition comprises one or more disintegrants. In certain embodiments, the disintegrant is selected from alginic acid, calcium alginate, carboxymethylcellulose calcium, carboxymethylcellulose sodium, cellulose, chitosan, colloidal silicon dioxide, corn starch, croscarmellose sodium, crospovidone, docusate sodium, glycine, guar gum, hydroxypropyl cellulose, magnesium aluminum silicate, methylcellulose, microcrystalline cellulose, pre-gelatinized starch, polacrilin potassium, povidone, silicates, sodium aliginate, sodium carbonate, and sodium starch glycolate.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof.

In some embodiments, the pharmaceutical composition comprises one or more surfactants. In some embodiments, the surfactant is selected from polyoxyethylene (20) sorbitan monolaurate (e.g., Tween-20), polyoxyethylene (20) sorbitan monooleate (e.g., Tween-80), sodium lauryl sulfate, and sodium dodecyl sulfate.

In some embodiments, the pharmaceutical composition comprises one or more pore formers. In some embodiments, the pore former is selected from hydroxypropylcellulose, hydroxypropylmethylcellulose, polyethyleneglycol, polaxamer 188, povidone (e.g., Kollidon K25/K30), or sugar (e.g., glucose, mannose, fructose, and sucrose).

In some embodiments, the pharmaceutical composition comprises one or more glidants. In some embodiments, the glidant is selected from calcium phosphate, cellulose, colloidal silicon dioxide, fumed silica, magnesium oxide, magnesium silicate, magnesium stearate, magnesium trisilicate, and talc. Suitable glidants include, but are not limited to, colloidal silicon dioxide, CAB-O-SIL™ (Cabot Co. of Boston, MA), and asbestos-free talc.

In some embodiments, the pharmaceutical composition comprises one or more lubricants. In some embodiments, the lubricant is selected from calcium stearate, glycerin monosterate, glyceryl behenate, glyceryl palmitostearate, hydrogenated castor oil, hydrogenated vegetable oil, light mineral oil, myristic acid, poloxamer, polyethylene glycol, sodium benzoate, sodium chloride, sodium lauryl sulfate, sodium stearyl fumarate, solid polyethylene glycols, stearic acid, and talc.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof.

In some embodiments, the pharmaceutical composition comprises one or more film coating agents. In some embodiments, the film coating comprises a poly(vinyl alcohol) base.

In some embodiments, the film coating includes a coloring agent or pigment. In some embodiments, the film coating is Opadry II® such as Opadry III) yellow.

Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye.

Suitable flavoring agents include, but are not limited to, natural flavors extracted from plants, such as fruits, and synthetic blends of compounds which produce a pleasant taste sensation, such as peppermint and methyl salicylate.

Suitable sweetening agents include, but are not limited to, sucrose, lactose, mannitol, syrups, glycerin, and artificial sweeteners, such as saccharin and aspartame.

Suitable emulsifying agents include, but are not limited to, gelatin, acacia, tragacanth, bentonite, and surfactants, such as polyoxyethylene sorbitan monooleate (Tween-20), polyoxyethylene sorbitan monooleate 80 (Tween-80), and triethanolamine oleate.

Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone.

Suitable preservatives include, but are not limited to, glycerin, methyl and propylparaben, benzoic acid, sodium benzoate, and alcohol.

Suitable wetting agents include, but are not limited to, propylene glycol monostearate, sorbitan monooleate, diethylene glycol monolaurate, and polyoxyethylene lauryl ether.

Suitable solvents include, but are not limited to, glycerin, sorbitol, ethyl alcohol, and syrup.

Suitable non-aqueous liquids utilized in emulsions include, but are not limited to, mineral oil and cottonseed oil.

Suitable organic acids include, but are not limited to, citric and tartaric acid.

Suitable sources of carbon dioxide include, but are not limited to, sodium bicarbonate and sodium carbonate.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

A tablet dosage form can be prepared from the active ingredient in powdered, crystalline, or granular forms, alone or in combination with one or more carriers or excipients described herein, including binders, disintegrants, controlled-release polymers, lubricants, diluents, and/or colorants.

A tablet of the present disclosure can be formulated for rapid, sustained, extended, or modified release.

In some embodiments, a unit dosage form of the invention comprises one or more pharmaceutically acceptable excipients selected from microcrystalline cellulose, lactose monohydrate (modified), croscarmellose sodium, hydroxypropyl cellulose, and magnesium stearate.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

For oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrants include, without limitation, starch, methyl cellulose, agar, bentonite, xanthan gum starches, agar, alginic acid or its sodium salt, or effervescent mixtures, croscarmellose or its sodium salt, and the like. Diluents include, e.g., lactose, dextrose, sucrose, mannitol, sorbitol, cellulose and/or glycine.

Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A therapeutically effective dose, of a compound described herein in an oral formulation, may vary from 0.01 mg/kg to 50 mg/kg patient body weight per day, more particularly 0.01 to 10 mg/kg, which can be administered in single or multiple doses per day. For oral administration, the drug can be delivered in the form of tablets or capsules containing 1 mg to 500 mg of the active ingredient specifically, 1 mg, 5 mg, 10 mg, 20 mg, 50 mg, 100 mg, 250 mg, and 500 mg, or in the forms of tables or capsules containing at least 1%, 2%, 5%, 10%, 15%, 20%, 25%, 30%, 40%, 50% (w/w) of the active ingredient. For example, the capsules may contain 50 mg of the active ingredient, or 5-10% (w/w) of the active ingredient. For example, the tablets may contain 100 mg of the active ingredient, or 20-50% (w/w) of the active ingredient. For example, the tablet may contain, in addition to the active ingredient, a disintegrant or emollient (e.g., croscarmellose or its sodium salt and methyl cellulose), a diluent (e.g., microcrystalline cellulose), and a lubricant (e.g., sodium stearate and magnesium stearate). The drug can be administered on a daily basis either once, twice or more per day.

Parenteral formulations comprising a compound described herein can be prepared in aqueous isotonic solutions or suspensions, and suppositories are advantageously prepared from fatty emulsions or suspensions. The formulations may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional methods, and may contain about 0.1 to 75%, preferably about 1 to 50%, of a compound described herein.

The phrases “parenteral administration” and “administered parenterally” are art-recognized terms, and include modes of administration other than enteral and topical administration, such as by injection, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

In some embodiments, the compound or pharmaceutically acceptable salt thereof is administered systemically. In some embodiments, the compound or pharmaceutically acceptable salt thereof is administered orally as part of a solid pharmaceutical composition. In some embodiments, the pharmaceutical composition is a liquid. In some embodiments, the pharmaceutical composition is administered as a liquid via nasogastric tube.

In some embodiments, the dose of the compound or pharmaceutically acceptable salt thereof is about 10 mg to about 10,000 mg per day. In some embodiments, the dose of the compound or pharmaceutically acceptable salt thereof is about 10 mg to about 7500 mg per day. In some embodiments, the dose of the compound or pharmaceutically acceptable salt thereof is about 50 mg to about 3600 mg per day. In some embodiments, the dose of the compound or pharmaceutically acceptable salt thereof is about 250 mg to about 2400 mg per day. In some embodiments, the dose of the compound or pharmaceutically acceptable salt thereof is about 600 mg to about 5000 mg per day. In some embodiments, the dose of the compound or pharmaceutically acceptable salt thereof is about 1000 mg to about 7500 mg per day.

In some embodiments, the compound or pharmaceutically acceptable salt thereof is administered once, twice, thrice, or four times per day. In some embodiments, the compound or pharmaceutically acceptable salt thereof is administered twice per day.

In some embodiments, the dose of the compound or pharmaceutically acceptable salt thereof is about 600 mg BID (i.e., twice per day); 1.2 g BID; or 2.4 g BID.

In some embodiments, the pharmaceutical composition is administered daily in one or more divided doses. In some embodiments, the composition is administered once per day (qua diem; QD). In some embodiments, the composition is administered twice per day (bis in die; BID). In some embodiments, the composition is administered thrice per day (ter in die; TID). In some embodiments, the composition is administered four times per day (quarter in die; QID). In some embodiments, the composition is administered every four (4) hours (quaque four hours; q4 h).

In some embodiments, compound I-1 is selected as the active pharmaceutical ingredient and is processed and manufactured to a solid form thereof, such as its most stable polymorph, prior to compounding into drug product. In some embodiments, the solid form, e.g., polymorph, is one of those described in PCT/US2020/031138, published as WO 2020/223685, hereby incorporated by reference.

In some embodiments, the solid form of compound I-1 is substantially amorphous or crystalline, or is a mixture thereof. In some embodiments, the solid form is substantially free of impurities.

In certain embodiments, compound I-1 is a crystalline solid. In some embodiments, compound I-1 is a crystalline solid substantially free of amorphous compound I-1. As used herein, the term “substantially free of amorphous compound I-1” means that the compound contains no significant amount of amorphous compound I-1. In some embodiments, at least about 95% by weight of crystalline compound I-1 is present. In still other embodiments of the invention, at least about 99% by weight of crystalline compound I-1 is present.

It has been found that compound I-1 can exist in a variety of solid forms. Exemplary such forms include polymorphs such as those described below.

In some embodiments, compound I-1 is amorphous. In some embodiments, compound I-1 is amorphous, and is substantially free of crystalline compound I-1.

In some embodiments, compound I-1 is a solid form in PCT/US2020/031138 selected from Compound 5:

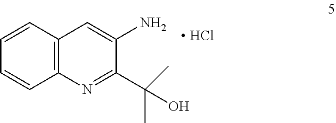

wherein the form is of Form A or Form B. In some embodiments, the compound is crystalline. In some embodiments, the compound is a crystalline solid substantially free of amorphous compound 5. In some embodiments, the compound is substantially free of impurities. In some embodiments, the compound has one or more peaks in its XRPD selected from those at about 13.9, about 15.8 and about 24.3 degrees 2-theta. In some embodiments, the compound has at least two peaks in its XRPD selected from those at about 13.9, about 15.8 and about 24.3 degrees 2-theta. In some embodiments, the compound is of Form A. In some embodiments, the compound has an XRPD substantially similar to that depicted in FIG. 21 of PCT/US2020/031138. In some embodiments, the compound has one or more peaks in its XRPD selected from those at about 10.2, about 17.0 and about 28.8 degrees 2-theta. In some embodiments, the compound has at least two peaks in its XRPD selected from those at about 10.2, about 17.0 and about 28.8 degrees 2-theta. In some embodiments, the compound is of Form B. In some embodiments, the compound has an XRPD substantially similar to that depicted in FIG. 23.

In some embodiments, compound I-1 is a solid form in PCT/US2020/031138 selected from the group consisting of:

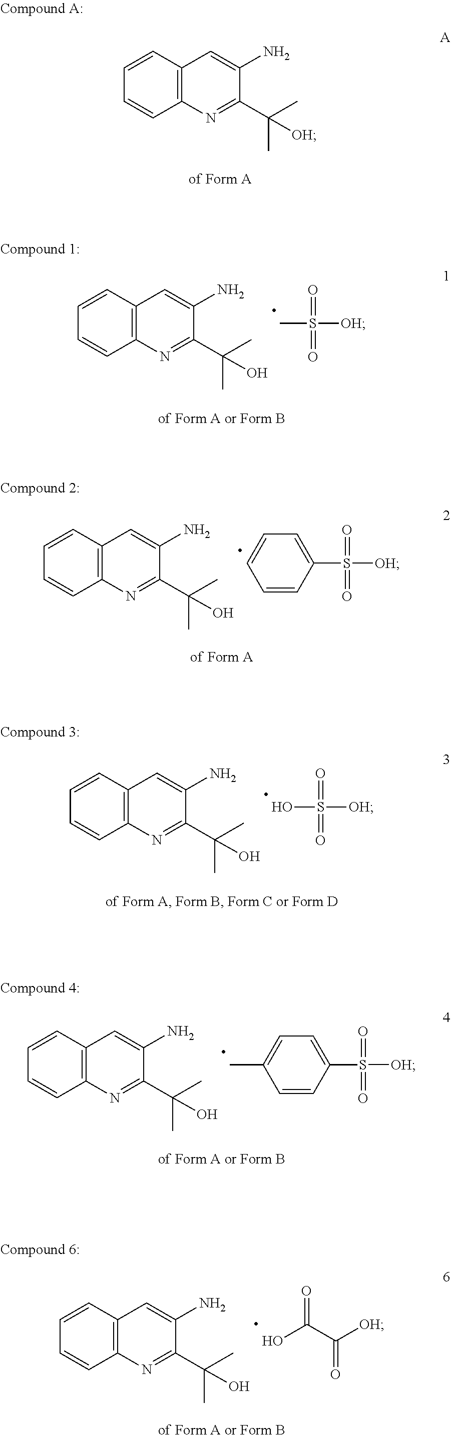

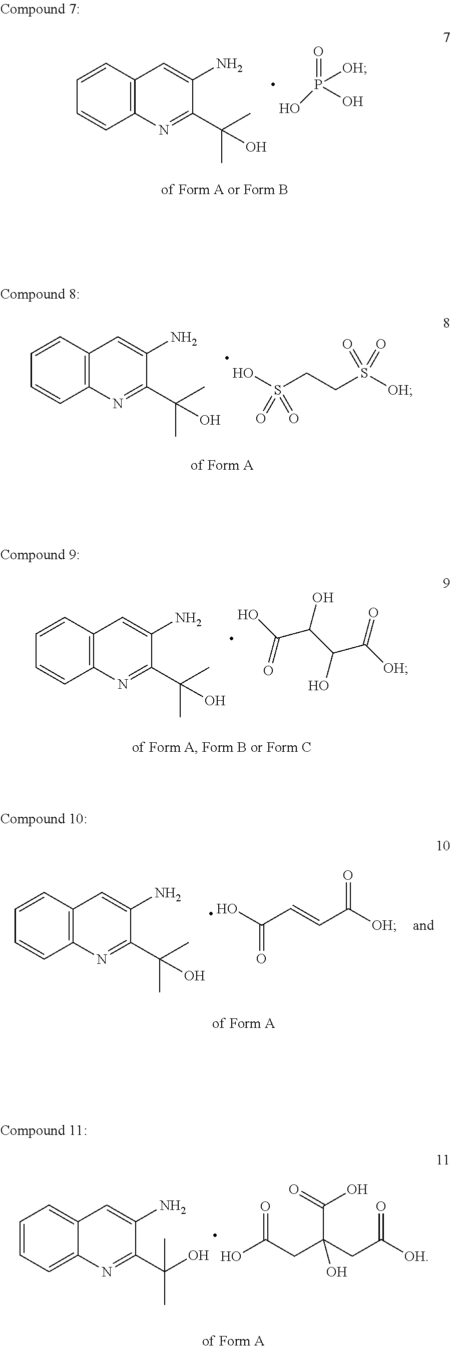

In some embodiments, the quinoline compound is a deuterium-enriched compound described in U.S. Pat. No. 10,550,085, the entirety of which is hereby incorporated by reference.

All publications, patents, patent applications and other documents cited in this application are hereby incorporated by reference in their entireties for all purposes to the same extent as if each individual publication, patent, patent application or other document were individually indicated to be incorporated by reference for all purposes.

All features of each of the aspects of the invention apply to all other aspects mutatis mutandis.

### EXEMPLIFICATION

### Example 1: An Open-Label, Phase 1/2, Single-Site Study of the Safety, Biochemical Efficacy, and Exploratory Clinical Effects of Oral Compound 1 in Subjects with Sjögren-Larsson Syndrome

### Introduction

Sjögren-Larsson syndrome (SLS) is an inherited disorder of fatty aldehyde metabolism characterized by clinical features of congenital ichthyosis, spastic diplegia, intellectual disability, seizures, and a distinctive retinopathy. The disease is caused by biallelic mutations in ALDH3A2, which results in deficient activity of fatty aldehyde dehydrogenase (FALDH) and leads to the build-up of harmful long-chain (C16-C20) aldehydes and alcohols. Accumulation of these lipids and their metabolic products in skin, brain, and eyes is responsible for the symptoms. The debilitating symptoms persist life-long, and there is no FDA-approved drug for SLS that addresses the pathogenic mechanisms.

Aldehydes are toxic molecules due to their propensity to covalently attach to proteins and other target molecules containing certain reactive chemical groups. Drugs that detoxify reactive aldehydes are under development for various inflammatory conditions characterized by the excessive formation of aldehydes. Aldehydes produced during inflammation typically have short- or medium-chain lengths (C3-C9), which do not accumulate in SLS. ADX-102 (Reproxalap, 2-[3-amino-6-chloro-quinolin-2-yl]-propan-2-ol) is an aldehyde trapping agent that has been shown to detoxify aldehydes. This drug shows beneficial activity as an ophthalmic preparation in Phase-2 clinical studies of allergic conjunctivitis and dry eye. Relevant to SLS, ADX-102 can block the formation of long-chain (C16-C18) fatty aldehyde adducts with cellular lipids, mitigate aldehyde cytotoxicity, and prevent metabolism of fatty aldehydes to fatty alcohols in cultured FALDH-deficient cells. When applied topically as a cream in a small double-blind, placebo-controlled study, ADX-102 was found to improve ichthyosis in SLS subjects. This drug cannot be administered orally due to its poor absorption qualities, which prevents its application for treating the non-cutaneous symptoms of SLS. In contrast, Compound 1 is well-absorbed orally and has undergone Phase 1 safety studies in normal controls. Compound 1 is anticipated to improve the biochemical abnormalities in SLS, while also explicitly targeting the systemic symptoms. This clinical trial will investigate the safety and biochemical efficacy of systemic administration of Compound 1 in SLS.

### Objectives:

The objectives of this study are to investigate the safety, tolerability, and biochemical efficacy of orally administered Compound 1 in subjects with SLS. Preliminary data will also be collected to look at potential short term clinical effects of Compound 1 on the neurologic, ophthalmologic and cutaneous symptoms of SLS.

### Study Design:

Tis is an open-label, single center study in which all subjects will be treated with Compound 1 for a period of 3 months.

### Number of Subjects Planned:

Up to 6 subjects with SLS will be studied.

### Inclusion Criteria:

Subjects meeting all of the following criteria will be considered eligible for study entry:
1. Subject is aged 10- to 50-years of age at the time of enrollment.
2. Subject has a genetically-confirmed diagnosis of SLS with two pathogenic sequence variants in ALDH3A2.
3. Subject has active ichthyosis and neurologic symptoms of spasticity.
4. Subject is willing to suspend use of all topical creams 1 week before administration of study drug and for 1 week before the end of the study at 12 weeks.
5. Subject has not been treated with any experimental drug for 1 month before enrollment and will take no other experimental drugs during Compound 1 therapy.
6. Subject is not sexually active or is documented to be surgically sterile or postmenopausal.
7. If sexually active, subject agrees to use adequate contraception throughout the duration of the study, as follows:
   a. For sexually active women of child-bearing potential: Negative pregnancy test at Baseline and compliant with a medically-approved contraceptive regimen during the study
   b. For sexually-active men: Compliant with a contraceptive regimen during the study or documented to be surgically sterile.
8. Subject or subject's guardian is willing and able to provide written informed consent prior to the initiation of any study procedures. Assent will be solicited from subjects intellectually capable of providing assent.

### Exclusion Criteria:

1. Subject has a history of malignancy.
2. Subject is known to be human immunodeficiency virus (HIV) positive or has other known immunodeficiency.
3. Subject has evidence of an active infection.
4. Subject has a history of any other condition that, in the opinion of the Investigator, would compromise the subject's ability to comply with the protocol or that might compromise the subject's safety or the interpretation of the study results.
5. Subject is currently receiving immunosuppressive therapy, including intermittent or low-dose corticosteroids and is not able or willing to suspend its use for a period from 2 weeks before the study and throughout the study.
6. Subject is currently receiving systemic or topical retinoids, other topically applied drugs, or any oral drugs or supplements that could interfere with dermatologic examination findings.
7. Subject receives an investigational systemic or topically administered drug within 30 days before enrollment.
8. Subject is currently taking and is unwilling or unable to discontinue any medication excluded in Prohibited Medications and Procedures.
9. Subject has a known allergic reaction to any ingredients of study drug formulation.
10. Subject is currently participating in any other therapeutic clinical study.
11. Subject is pregnant.

### Drag Dose and Mode of Administration:

All subjects will be treated orally with Compound 1 at a dose of 250 mg BID.

### Duration of Treatment:

Subjects will be treated with study drug for 3 months.

### Study Endpoints:

The primary endpoint is safety. Secondary endpoints will include measures of biomarkers. Exploratory evidence for initial clinical response to Compound 1 will also be sought.

### Primary Endpoint

Safety will be determined by documentation of adverse events (AEs), including serious adverse events (SAEs), and clinical laboratory tests (hematology, clinical chemistries, urinalysis, pyridoxal phosphate, retinal and retinol). The parameters derived from these safety variables are:
   a. The proportion of subjects experiencing AEs, SAEs, and AEs leading to discontinuation of study drug.
   b. Clinically significant changes in laboratory tests.

### Secondary Endpoints

Biomarkers: Plasma specimens will be analyzed for metabolomics/lipidomics, fatty alcohols, plasma alkylglycerols, and oxidative stress biomarkers (oxysterols, 4-hydroxy-2-nonenal, glutathione).
Superficial skin (stratum corneum) will be collected for lipid analysis of fatty alcohols, alkylglycerols, oxysterols, cholesterol, and cholesterol precursors.

### Exploratory Clinical Activity

The potential clinical effects of Compound 1 will focus on the known clinical symptoms of SLS. Measurements of drug activity will search for changes from Baseline (week-1) to last week on study drug (week-12):
Clinical severity of ichthyosis using the Visual Index of Ichthyosis Severity (VIIS) and skin digital photography.
Measurements of skin thickness and elasticity using high frequency Ultrasound with Shear-Wave Elastography (US-SWE).
Neurologic changes in brain disease using clinical spasticity scores (Modified Ashworth Spasticity score, GMFM, 6-minute walk test, Berg balance scale/pediatric balance scale, and Timed Up and Go/Timed Floor to Stand), brain magnetic resonance imaging (MRI), magnetic resonance spectroscopy (MRS), and electroencephalogram (EEG).
Ophthalmologic changes in visual acuity, optical coherence tomography (OCT), and retinopathy using image analysis of retinal photographs.
Quality of Life (QOL) score using the Promis QOL survey: IQoL-32 Ichthyosis QOL survey; and a Parent/Subject Satisfaction survey.

Statistical Methods: Statistical analyses of safety will be primarily descriptive in nature with summary of adverse events (AE) and serious adverse events (SAE). Biomarker data at baseline (week-1) and week-12 will be compared using paired t-tests with significance at p<0.05.

Continuous variables will be summarized using descriptive statistics [n, mean, standard deviation, median, minimum, and maximum]. Categorical variables will be summarized showing the number and percentage (n, %) of subjects within each classification.

### Schedule of Events

| | | | | |
|---|---|---|---|---|
| | | Week-4 | Week-8 | |
| | Week-1 | Local | Local | Week-12 |
| | Site | Telehealth | Telehealth | Site |
| Evaluation | Visit | Visit | Visit | Visit |

| | | |
|---|---|---|
| Written consent ^{a} | X | |
| Physical exam ^{b} | X | X |
| Pregnancy test ^{c} | X | |

Safety Tests

| | | | | |
|---|---|---|---|---|
| CBC with diff ^{d} | X | X | X | X |
| CMP ^{e} | X | X | X | X |
| Urinanalysis ^{f} | X | X | X | X |
| Vitamin A ^{g} | X | X | X | X |
| Pyridoxal phosphate ^{h} | X | X | X | X |
| Homocysteine ⁱ | X | X | X | X |
| ECG ^{j} | X | | | X |

Adverse Events will be collected on weekly telephone calls ^{z}
Research Tests and Evaluations

| | | |
|---|---|---|
| Plasma metabolomics, | X | X |
| lipidomics ^{k} | | |
| Plasma fatty alcohols ^{l} | X | X |
| Plasma alkylglycerols ^{m} | X | X |
| Plasma oxid. stress | X | X |
| markers ⁿ | | |
| Skin lipids ^{o} | X | X |
| Plasma Compound 1 | X | X |
| level and archive | | |
| research blood ^{p} | | |
| MRI/MRS ^{q} | X | X |
| EEG ^{r} | X | X |
| Spasticity scores ^{s} | X | X |
| Cutaneous | X | X |
| ultrasound/TEWL ^{t} | | |
| Skin photos/VIIS ^{u} | X | X |
| Ophthalmologic exam | X | X |
| (visual acuity, retinal | | |
| photos, OCT) ^{v} | | |
| Promis QOL survey ^{w} | X | X |
| IQoL-32 Ichthyosis | X | X |
| Quality of Life ^{x} | | |
| Subjective survey ^{y} | X | X |

^{a} Written consent will be obtained from both parents, or the LAR if only one parent has legal custody of a minor subject.
^{b} Physical examination will include neurologic exam and dermatologic exam.
^{c} For female subjects of child-bearing potential and who are sexually active.
^{d} Hematology parameters to be measured included red blood cell count, hemoglobin, hematocrit, platelet count, white blood cell count, and differential (neutrophils, lymphocytes, monocytes, eosinophils, and basophils).
^{e} Comprehensive metabolic panel (CMP) includes sodium, potassium, chloride, bicarbonate, blood urea nitrogen, calcium, creatinine, glucose, albumin, alkaline phosphatase, aspartate aminotransferase, alanine aminotransferase, total bilirubin, and total protein.
^{f} Urinalysis includes specific gravity, pH, glucose, protein, ketones, esterase, nitrites, and cytology for cells.
^{g} Vitamin A consists of plasma retinal and retinol.
^{h} Plasma level of pyridoxal phosphate.
ⁱ Plasma homocysteine is a functional marker for pyridoxal phosphate deficiency.
^{j} Standard 15-lead ECG.
^{k} Plasma metabolomics/lipidomics for SLS biomarker panel.
^{l} Plasma fatty alcohols (C16-C18) measured by GC-MS.
^{m} Plasma alkylglycerol lipids measured by GC-MS.
ⁿ Plasma oxidative stress markers include oxysterols, 4-hydroxynonenal, and glutathione (GSH).
^{o} Skin cell samples will be collected from the stratum corneum by tape stripping with D-Squames for cholesterol, sterols and alkylglycerol measurements.
^{p} Blood (5 ml) will be collected for Compound 1 level, frozen and archived for potential use, if needed.
^{q} MRI/MRS will be done under anesthesia, if necessary.
^{r} Conventional EEG
^{s} Spasticity scoring will include Modified Ashworth Spasticity Score, Gross Motor Function Measure (GMFM), 6-minute walk test, Berg Balance Scale/Pediatric Balance Scale, and Timed Up and Go/Timed Floor to Stand testing.
^{t} Cutaneous ultrasound with elasticity measurements will quantitate skin thickness and elasticity. Transepidermal water loss (TEWL) is a functional measure of the epidermal water barrier.
^{u} Skin photos are obtained for scoring of ichthyosis using Visual Index of Ichthyosis Severity (VIIS).
^{v} Ophthalmologic exam will include optical coherence tomography (OCT) to visualize the retina, and retinal photos for image analysis to detect changes in macular crystalline inclusions.
^{w} The age-appropriate Promis Quality of Life survey will be completed.
^{x} The IQoL-32 Ichthyosis Quality of Life survey will be administered.
^{y} A SLS Subjective survey.
^{z} A follow-up telephone call will be made to the subject and/or caregiver once weekly throughout the study to inquire about Adverse Events.

### List of Abbreviations

AE, adverse events
ALDH3A2, aldehyde dehydrogenase 3A2
ALP, alkaline phosphatase
ALT, alanine aminotransferase
AST, aspartate aminotransferase
AUC, area under the curve
BID, twice per day
Botox, botulinum toxin
BSA, body surface area
CBC, complete blood count
CHRI, Child Health Research Institute
CMP, comprehensive metabolic panel
CRF, case report form
CTCAE, Common Terminology Criteria for Adverse Events
CYP, cytochrome P450 enzyme
DSMB, Data Safety Monitoring Board
ECG, electrocardiogram
EEG, electroencephalogram
FALDH, fatty aldehyde dehydrogenase
FMDA, free malondialdehyde
GC-MS, gas chromatography-mass spectrometry
GGT, gamma glutamyltransferase
GMFM, Gross Motor Function Measure
GSH, glutathione
HIV, human immunodeficiency virus
ICF, informed consent form
IRB, Institutional Review Board
LC/MS-MS, liquid chromatography/tandem mass spectrometry
MRI, magnetic resonance imaging
MRS, magnetic resonance spectrometry
MAD, multiple ascending dose
MDA, malondialdehyde
MedDRA, Medical Dictionary for Regulatory Activities
NAPE, N-alkyl-phosphatidylethanolamine (aldehyde adduct with PE)
NCI, National Cancer Institute
NOAEL, no-observed-adverse-effect level
OCT, ocular coherence tomography
PE, phosphatidylethanolamine
PI, Principal Investigator
PK, pharmacokinetics
PT, preferred term
QOL, Quality of Life
RASP, reactive aldehyde species
SAE, serious adverse events
SAD, single ascending dose
SLS, Sjögren-Larsson syndrome
SOC, system organ classification
TEAE, treatment-emergent adverse effect
TEWL, transepidermal water loss
UA, urinalysis
US-SWE, Ultrasound with Shear-Wave Elastography
VIIS, Visual Index of Ichthyosis Severity
VLC, very long-chain

### BACKGROUND

Sjögren-Larsson syndrome (SLS) is a rare, inherited disease characterized by congenital ichthyosis, spasticity, intellectual disability, and a distinctive retinopathy^{1,2,3}. Since its first description in Sweden more than 60 years ago⁴, SLS has been seen worldwide and in all ethnic groups⁵. The syndrome has an overall estimated prevalence of 1:250,000 and is inherited in an autosomal recessive manner. Although rare, the disease is the most widely recognized form of neuro-ichthyosis⁶. In some embodiments, the method improves seizures, spasticity, intellectual disability, retinopathy, neuro-ichthyosis.

The ichthyosis in SLS is apparent at birth and becomes more established by several months of age^{2,7}. It is generalized in distribution and particularly prominent in the flexure areas, nape of the neck, trunk, and extremities. A mild erythema is often present. Pruritus is an agonizing skin symptom in most patients and can lead to bleeding excoriations.

The neurologic symptoms develop within the first 2 years of life and usually prompt the recognition of SLS rather than another type of ichthyotic disorder². Spastic diplegia is typically present and much more common that spastic quadriplegia, which denotes a more severe phenotype⁸. Spasticity is associated with delayed motor milestones, such as sitting, crawling and standing, and interferes with ambulation. Patients require walkers, crutches or canes to walk and most are fitted with ankle-foot orthotic supports. Seizures occur in 40% of patients¹. Cognitive impairment tends to be initially mild, but worsens during mid-childhood and culminates in moderate to severe intellectual disability.

Ophthalmologic features of SLS include variable myopia, photophobia, and macular degeneration with characteristic perifoveal glistening white dots^{9,10}. These crystalline deposits first appear in childhood and increase over time¹¹. Longitudinal studies, however, indicate that the retinal dots are dynamic structures that can appear and sometimes later disappear¹².

Although the typical clinical phenotype of SLS is well known, patients with a more severe neurodegenerative phenotype¹³ or with milder cases¹⁴ have been reported. Symptoms of SLS persist throughout life and most patients survive into adulthood.

Genetics and Biochemical Pathogenesis: SLS is caused by mutations in the ALDH3A2 gene that encodes fatty aldehyde dehydrogenase (FALDH)^{15,16}. More than 130 different mutations have been reported in SLS patients including amino acid substitutions, small deletions, insertions, splicing defects and large contiguous gene deletions¹⁷. Missense mutations account for about one-third of all variants and most result in complete loss of FALDH activity¹⁶. Most mutations (60%) are private, but several common mutations have been reported throughout the world. To date, no clear genotype-phenotype correlations are known.

In SLS, ALDH3A2 mutations result in deficient activity of FALDH, a microsomal enzyme that oxidizes fatty aldehydes to fatty acids¹⁸. FALDH is also a component of a multiprotein complex (fatty alcohol: NAD⁺ oxidoreductase) that oxidizes fatty alcohols to fatty acids via a fatty aldehyde intermediate¹⁹. Therefore, FALDH deficiency results in impaired oxidation of both long-chain aldehydes and alcohols. Both lipid substrates and their metabolic by-products are implicated in disease symptoms. Fatty aldehydes are a degradation product of several lipids, including ether lipids, sphingolipids, isoprenols (farnesol, geranylgeraniol), and phytanic acid leukotriene B4 and 2-OH-very long-chain fatty acids²⁰. Tissues differ in the synthesis, content, and degradation of these aldehydogenic lipids, which may account in part for the symptom distribution²¹. Of note, free fatty aldehydes do not accumulate appreciably in SLS cells, due to their highly reactive nature and propensity to form covalent aldehyde adducts with membrane proteins and certain lipids containing free amino groups (i.e. phosphatidylethanolamine, PE). Alternately, fatty aldehydes are metabolized to fatty alcohols by the action of aldehyde reductase, particularly when aldehyde oxidation is deficient in SLS due to FALDH deficiency²².

Fatty alcohols and their metabolic by-products (ether lipids; wax esters) accumulate in SLS patients' plasma, skin and brain^{23,24,25}. In SLS skin, fatty alcohols (C16-C20) that cannot be metabolized to fatty acids are instead diverted into an excessive synthesis of wax esters and ether glycerolopids²⁶. These lipids disrupt the synthesis and secretion of lamellar bodies, which are cytosolic vesicles that normally carry membranes to the plasma membrane and, via exocytosis, contribute their membrane cargo to the stratum corneum for formation of the epidermal water barrier. Rather than membrane cargo, many lamellar bodies in SLS are empty or contain granular material²⁷. Consequently, there is a paucity of stratum corneum membranes, which also accumulate abnormal non-membranous lipid deposits. As a result, the water barrier is disrupted, prompting a reactive hyperproliferative response resulting in excess scaling and the clinical appearance of ichthyosis²⁷.

Uniquely in brain, accumulation of fatty alcohols with an unusual very long chain (VLC) profile (C21-C24) and their corresponding VLC-ether lipids occurs²⁵. These VLC-alcohols are thought to arise from alpha-oxidation of 2-OH-VLC fatty acids, which generates VLC-aldehydes that cannot be further oxidized to fatty acids due to FALDH deficiency. Instead, the VLC-aldehydes are diverted to formation of VLC-alcohols and incorporated into VLC-ether lipids, which can be detected in SLS subjects as abnormal lipid peaks on magnetic resonance spectroscopy (MRS)^{28,29}, and probably accounts for the myelin abnormalities seen on magnetic resonance imaging (MRI)³⁰. It is therefore likely that diversion of VLC-aldehydes to VLC-alcohols and VLC-ether lipids in the brain is a key mechanism for neurologic disease in SLS²⁵, possibly augmented by formation of covalent VLC-aldehyde adducts with myelin basic protein and/or phosphatidylethanolamine (NAPE) lipid. In addition, myelin membranes have an unusually high proportion (85%) of plasmalogen PE (ether phospholipid), which produces fatty aldehydes when these ether lipids are degraded, adding to the aldehyde load.

Current SLS Therapy: Due to its rarity, few pathogenesis-based interventions have been studied in SLS subjects²¹, and there are no disease-specific FDA-approved drugs for the management of SLS. Currently, only non-specific generic therapies are available. Topical moisturizing creams are used to form a temporary water barrier; keratolytic agents (salicylic acid, lactic acid, urea) help remove scales; and daily water baths help hydrate the skin. Rarely, systemic retinoids are used to improve the ichthyosis. Neurologic symptoms are treated non-specifically. Spasticity is improved temporarily with Botox injections but most patients are eventually treated with surgical procedures such as tendon lengthening or dorsal root rhizotomy. Intrathecal baclofen has recently been reported to improve spasticity in two patients ³¹. Seizures usually respond to anticonvulsants. Although some fatty aldehydes and alcohols are present in the diet, they do not contribute materially to overall lipid content in SLS, since low-fat diets do not have a significant impact on plasma fatty alcohols or clinical severity³².

### SLS Biomarkers:

To monitor efficacy of Compound 1 in SLS trials, several potential biomarkers have been identified.

Brain MRI demonstrates white matter disease, and MRS reveals abnormal lipid peaks with resonances at 1.3 ppm and 0.9 ppm^{28,29}.

Electroencephalography (EEG) demonstrates generalized slow wave abnormalities, even in subjects without a history of seizures.

Plasma fatty alcohols (C16-C18) are elevated in SLS²³.

Metabolomic studies in plasma of SLS subjects identifies a unique panel of “SLS biomarkers” including elevated sphingosine and sphingosine-1-phosphate, reduced sphingomyelin, abnormal reduction in 3-OH-5-cholestenoic acid, elevation of 4-cholestene-3-one, elevated serotonin, and reduced bile acids (Dai and Rizzo, unpublished observations). Oxidative stress biomarkers are also increased.

Cutaneous scales (stratum corneum) have a unique lipid signature comprised of abnormal accumulation of neutral ether lipids (alkyl-diacylglycerol), cholesterol, and fatty alcohols. Neutral ether lipids are increased up to 100-fold in SLS compared to control skin²⁴, and cholesterol is increased 5-fold (Korade and Rizzo, unpublished).

Cutaneous ultrasound with shear-wave elastography demonstrates increased epidermal thickness and reduced elasticity (stiffness) in skin (Li and Rizzo, unpublished observations).

Transepidermal water loss (TEWL) is increased in SLS as measured using a Tewameter surface probe.

Urinary leukotriene B4, which requires FALDH for its degradation, is increased in SLS subjects³³.

### Rationale for Systemic Compound 1 in SLS

The rationale for this Compound 1 drug trial is based on the following points.

The proposed mechanism for neurologic disease in SLS is that VLC-aldehydes, which cannot be oxidized due to FALDH deficiency, are diverted to excessive formation of VLC-alcohols and VLC-ether lipids. Accumulation of these lipids, which are detected on MRS as lipid peaks in brain white matter, disrupts myelin stability and contributes to spasticity and intellectual disability development.

Without wishing to be bound by theory, it is believed that Compound 1 interferes with the aldehyde-dependent pathogenic mechanisms in SLS and mitigate the debilitating symptoms.

There are no satisfactory animal models of SLS, thereby limiting preclinical testing to in vitro cellular studies. In cultured Chinese hamster cells lacking FALDH³⁴, compound ADX-102 (also known as reproxalap) prevents fatty aldehydes from forming covalent adducts with PE (NAPE)³⁵. ADX-102 reverses fatty aldehyde cytotoxicity and apoptosis. It also blocks the metabolism of fatty aldehyde (C18:0, octadecanal) to fatty alcohol, while having no effect on synthesis or degradation of fatty alcohol itself (data not shown). The detoxified ADX-102-fatty aldehyde by-products are degraded intracellularly. Thus, treatment of cultured FALDH-deficient cells with ADX-102 bypasses the lost catabolic function of FALDH on aldehydes in SLS and diminishes aldehyde load.

### Drug Product

Compound 1 drug product is manufactured and supplied as 250 mg unit dose tablets for oral delivery. The drug product is packaged as 30 capsules in a 60 cc high-density polyethylene bottle.

Compound 1 drug product must be stored at 2° C. to 8° C. (35.6° F. to 46.4° F.) in an area with access limited to the investigator and designated personnel. The Compound 1 drug should not be frozen.

### Study Objectives

### Primary Objective

The primary objective of this study is to evaluate the safety and tolerability of oral Compound 1 in subjects with SLS.

### Secondary Objectives

The secondary objectives of the study include:
Determine the effects of Compound 1 on relevant SLS metabolic biomarkers including metabolomic/lipidomic profile, fatty alcohols, fatty aldehydes, aldehyde adduct N-alkyl-phosphatidylethanolamine, ether lipids, lipid signature in blood and skin.

### Exploratory Objectives

The exploratory objectives of this study are to:
Evaluate the short term neurologic effects of Compound 1 on brain MRI, MRS, EEG and spasticity.
Evaluate and compare pre- and post-treatment cutaneous disease biomarkers in skin.

### Study Endpoints

### Primary Endpoints

Safety will be determined by documentation of adverse events (AEs) and clinical laboratory assessments. The endpoints derived from these safety variables are:
The proportion of subjects experiencing an AE;
The proportion of subjects experiencing a serious adverse event (SAE);
The proportion of subjects experiencing an AE leading to discontinuation of study drug.
Clinically significant changes in laboratory safety tests (CBC, CMP, UA, pyridoxine phosphate, homocysteine, vitamin A).

Tolerability will be determined by the compliance of subjects to take Compound 1 as measured by subject report and capsule count remaining after return of unused drug to the PI.

### Secondary Endpoints

Biochemical responses to Compound 1 will be evaluated by comparing baseline vs 12-week measures of:
Plasma metabolomic/lipidomic profile.
Plasma fatty alcohols, fatty aldehydes, and aldehyde adduct N-alkyl-phosphatidylethanolamine.
Alkylglycerol, fatty alcohol and cholesterol content of cutaneous scales.
Plasma oxidative stress markers including oxysterols, glutathione (GSH), malondialdehyde, and 4-hydroxynonenal.

### Exploratory Endpoints

The exploratory endpoints will investigate potential early clinical response to Compound 1. These will focus on the nervous system, eye and skin, the organs most impaired in SLS. Baseline and 12-week results will be compared to search for drug effects.
Clinical measures of spasticity (Modified Ashworth Spasticity Scale, Gross Motor Function Measure (GMFM), 6-minute walk test, Berg Balance Scale/Pediatric Balance Scale, and Timed Up and Go/Timed Floor to Stand testing).
Brain magnetic resonance imaging (MRI) and magnetic resonance spectroscopy (MRS).
Conventional EEG.
Cutaneous thickness and elastic properties as measured by Ultrasound with Shear Wave Elastography (US-SWE).
Cutaneous transepidermal water loss as measured using a Tewameter instrument.
Ichthyosis severity using the Visual Index of Ichthyosis Severity (VIIS).
Ophthalmologic examination for visual acuity, optical coherence tomography, and retinal photographs. Photographs will be submitted to image analysis to detect changes in macula and glistening white dots.
Promis Quality of life survey; IQoL-32 Ichthyosis survey.
Subjective survey of clinical response by subject and/or caregiver.

### Overall Study Design

This trial is an open-label, Phase 1/2, single-site study to evaluate the safety, tolerability, biochemical efficacy, and exploratory clinical effects of Compound 1 administered daily to subjects with SLS.

Study description: All study evaluations and tests will be done as outpatient visits. Screening interviews and written consents will be obtained within 30 days before drug administration. To evaluate the effects of study drug on the ichthyosis in its natural state and minimize contribution of exogenous topical lipids to cutaneous biochemical measurements, subjects will be instructed to withhold use of any topical lotions for one week prior to baseline study visit and one week before the final 12-week visit. They will be allowed to apply topical lotions between visits to the study center. Subjects will also be instructed to not bathe for 12 hours before study visits.

The schedule of visits for the study is provided above. Upon enrollment, all subjects will be evaluated at the study center. Subjects will be administered study drug only after routine baseline safety tests (CBC, comprehensive chemistry panel, UA) are completed and biospecimens collected on Day 1 or Day 2 of Week-1. On Day 2 of Week-1, Compound 1 will be administered to subjects. On Day-3, subjects will return to the study center for initial follow up evaluation and inquiry about drug tolerability and any adverse responses. Subjects will be discharged home to continue Compound 1 administration. A weekly phone call will be made by the Clinic Coordinator to the subject's parent/LAR between study center visits to inquire about potential adverse events. Safety blood tests will be collected locally at Week-4 and Week-8. Subjects will return to the study center at Week-12 for follow-up evaluation and repeat of all baseline clinical and laboratory tests. One week after completion of the Week-12 visit, the subject or parent will be contacted by the Study Coordinator via telephone for a follow-up safety inquiry.

### Recruitment and Selection of Study Population

Up to 6 subjects with SLS will be enrolled. These subjects will be identified from a larger cohort of SLS subjects known to the PI and followed at UNMC.

We expect to recruit SLS subjects nationwide. Subjects ages 10-50 years who meet eligibility criteria will be enrolled in the study. Males and females will be recruited. Due to the rare nature of SLS and the limited subject population, recruitment may not be equally divided by sex.

### Prohibited Concurrent Medications

Systemic retinoids such as acetretin (Soriatane), tretinoin (Retin-A, Altreno, Atralin, Avita, Biacna), isotretinoin (Accutane, Absorica, Claravis, Epuris, Sortret), tazarotene (Tazorac, Avage, Zorac), and alitretinoin (Panretin, Toctino).

Topical retinoids such as retinyl palmitate, retinaldehyde, retinol, or related drugs.

Any systemic or topically administered investigational drug within 30 days before Screening or during study participation.

Sensitive drug substrates of cytochrome P450 2C19 and 3A4.

Immunosuppressive therapy, including intermittent or low-dose corticosteroids.

Baclofen and botulinum toxin (Botox) injection during the study or within 6 months of enrollment.

### Permitted Concurrent Medications

Medications not specified above are permitted during the study for the treatment or prevention of disease or to maintain good health. Anticonvulsants used by the subjects for control of seizures are permitted. Vitamins (except Vitamin A, Vitamin B, and Vitamin D, including any multivitamin or supplement containing any or all), minerals, and dietary supplements are permitted at dosages considered by the PI as reasonable for maintaining good health.

If it becomes necessary for a subject to take a medication during the study, the specific medication(s) and indication(s) are to be discussed with the PI. All concomitant medications taken during the study are recorded in the source documents and CRF.

### Treatment Compliance

Subjects and/or caregivers will be given detailed instructions regarding the administration of study drug. A paper-based dosing diary will be dispensed for home use to record the date and time of each study drug administration and any missed doses. The reason for missed doses will be documented in the dosing diary, as well as in source documents and the CRF. Subjects and/or caregivers will be instructed to send the dosing diary to the study Coordinator every month. Study center personnel will review the information with the subject and/or caregiver. The Coordinator will also inquire about compliance during the weekly safety telephone call.

### Study Assessments

### Demographics and Medical History

Demographic information, including age, sex, race, and ethnicity, will be recorded during initial Screening. A complete medical history will also be documented during Screening, including date and/or age of diagnosis of SLS, genotype, and all prior medical and surgical treatments for SLS.

### Prior and Concomitant Medications

All prior and current medications will be documented at Screening and recorded in the subject's source documents and CRF.

### Physical Examination

A complete physical examination, including vital signs (pulse rate, respiratory rate, systolic and diastolic blood pressure, and body temperature), measurement of height, weight, and body surface area (BSA), will be conducted at baseline and at the 12-week visit. Results will be recorded in the CRF. The physical examination will include:

A dermatologic exam with photographs of the skin. The body site location of hyperkeratosis, scaling, erythema and excoriations will be recorded. Photographs will be obtained of the back, upper arms, lower legs, and dorsal feet for VIIS scoring, along with additional body sites.

A neurologic examination will document deep tendon reflexes, cranial nerves, tremors, dysmetria, dysarthria fine motor abilities including writing sample, and Romberg test.

Spasticity evaluation will include Modified Ashworth Spasticity score, Gross Motor Function Measure (GMFM), 6-minute walk test, Berg Balance Scale/Pediatric Balance Scale, and Timed Up and Go/Timed Floor to Stand testing.

### Laboratory Safety Evaluations

The following laboratory parameters will be measured to monitor safety:
Complete Blood Count (CBC): Hemoglobin, Hematocrit, White blood cell count with differential, Platelets, RBC indices.
Comprehensive Metabolic Panel: Sodium, Potassium, Chloride, Bicarbonate, Glucose, BUN. Total protein, Albumin, Bilirubin, Aspartate Aminotransferase (AST), Alanine Aminotransferase (ALT), Alkaline phosphatase.
Urinalysis: Specific gravity, pH, Glucose, Ketones, Protein, Nitrates, Esterase, Microscopic analysis for cells.
Plasma Pyridoxal-phosphate (vitamin B6): Because the potential exists for Compound 1 interfering with pyridoxal metabolism, plasma pyridoxal-phosphate will be measured by the Mayo Clinic Laboratory using LC/MS-MS. Subjects will be supplemented with vitamin B6 if necessary.
Plasma homocysteine: Pyridoxal-phosphate is a cofactor for many transaminase enzymes, including AST, ALT and cystathionine-B-synthase, which metabolizes homocysteine. Elevated plasma homocysteine is a sensitive marker for deficiency of pyridoxal-phosphate³⁶.
Plasma vitamin A (Retinal and retinol): The potential effect of Compound 1 on retinal metabolism will be monitored by measuring plasma vitamin A levels. Subjects will be supplemented with vitamin A, if levels of this vitamin decrease on Compound 1.
Electrocardiogram (ECG): A 15-lead ECG will be obtained at each clinic visit to look for any effects of Compound 1 on cardiac function in the SLS subjects.
Pregnancy: A urine pregnancy test will be performed for sexually active female subjects of childbearing potential during Screening. Results must be confirmed to be negative before the subject can be enrolled in the study. A urine pregnancy test will also be performed for female subjects of childbearing potential at Week 12, and at any time during the study if the subject reports the possibility of pregnancy.

### Biochemical and Exploratory Research Tests

Plasma lipidomics/metabolomics: SLS patients have a highly unique metabolomic profile that reflects abnormalities in several biochemical pathways.

Plasma fatty alcohols: SLS patients accumulate hexadecanol (C16:0 alcohol) and octadecanol (C18:0 alcohol) in plasma²³. These lipids are not detected with routine lipidomic methods.

Plasma and cutaneous alkylglycerol lipids: SLS subjects have increased plasma C18:0-alkylglycerol and elevated cutaneous alkylglycerols (C15-C20)²⁴.

Brain MRI/MRS: MRI typically reveals periventricular white matter disease in SLS patients by 5 years of age, and MRS demonstrates lipid accumulation in white matter.

EEG: Electroencephalography (EEG) reveals slowing of background electrical activity in most SLS subjects, consistent with generalized brain disease.

Spasticity testing: Spasticity in the arms and legs will be documented using several different but complementary functional tests.

Transepidermal water loss (TEWL): TEWL is abnormally increased in SLS subjects. A decrease in TEWL would therefore indicate effectiveness of the treatment.

Skin thickness and elasticity: Using cutaneous ultrasound, both parameters are abnormal in SLS and together provide a quantitative measure of ichthyosis severity. The test will constitute an anatomical correlate to TEWL.

Visual Index of Ichthyosis Severity (VIIS): The VIIS score is a clinically validated measure of ichthyosis severity³⁷.

Ophthalmologic examination: Testing of visual acuity will be done. Retinal photographs and optical coherence tomography (OCT) will document the retinal pathology in SLS subjects⁹. Image analysis of retinal photographs will identify changes in perimacular crystalline inclusions¹².

Promis Quality of Life survey: The NIH-funded Patient Reported Outcomes Measurement Information System is a popular survey tool that has been validated across multiple disciplines³⁸. There are age-dependent versions for pediatric and adult subjects.

IQoL-32 Ichthyosis Quality of Life survey: The IQoL-32 is a 32-question self-reported survey that is focused on the skin symptoms in SLS³⁹.

SLS Satisfaction survey: This is a general survey of subjects/caregivers' impressions of drug response.

### Biospecimen Collection

Fasting blood samples will be taken at Baseline (Day 1) and end of Week-12 for analysis of blood-based biomarkers. Safety blood and urine tests will be obtained at Weeks 4, 8 and 12. Standard phlebotomy procedures will be used. Safety blood tests will be processed and analyzed immediately. Research samples will be centrifuged and the plasma will be frozen at −70° for subsequent analysis.

Superficial stratum corneum skin cell samples will be collected from the right forearm and thigh using a 25-mm diameter skin sampling disc (e.g., D-SQUAME®). At least two discs will be collected from each site and stored at −70 degrees for subsequent batch analysis after Compound 1 drug administration has been completed for all subjects.

### Biomarker Measurements

Plasma metabolomics/lipidomics: Metabolomics and lipidomics will be performed using an LC/MS-MS platform. This method identifies and quantitates >2000 metabolites and lipids in plasma. Previous studies have demonstrated a highly unique and abnormal metabolomic profile for SLS consisting of lipid and non-lipid metabolites.

Plasma fatty alcohols: SLS subjects accumulate hexadecanol (C16:0 alcohol) and octadecanol (C18:0 alcohol) in blood²³. Plasma fatty alcohols will be measured using GC/MS as described²³. These lipids are not detected with routine lipidomic methods using LC/MS-MS.

Plasma and cutaneous alkylglycerol lipids: SLS subjects have increased plasma C18:0-alkylglycerol and highly elevated cutaneous alkylglycerols (C15-C20). Plasma and cutaneous alkylglycerol lipids will be measured by GC/MS as described²⁴. Lipids will be extracted from the stratum corneum cells adhered to the D-SQUAME using chloroform/methanol, hydrolyzed and alkylglycerols measured using single ion monitoring of fragment m/z 205. The method is sensitive and high reproducible (analytical CV of 7.2%).

Cutaneous cholesterol: The same cutaneous lipid extract used for measuring alkylglycerols will be used to measure cholesterol using an LC/MS-MS with a ThermoScientific TSQ Quantic triple-stage quadrupole mass spectrometer and deuterated internal standards as described⁴⁰. The analytical method has high sensitivity, dynamic range and reproducibility (CV<10%) for cholesterol and related sterol precursors.

Oxidative biomarkers: Plasma oxysterols will be measured by LC/MS-MS⁴⁰. 4-Hydroxynonenal will be measured in the Rizzo lab using GC/MS with deuterated internal standard. Glutathione will be measured together with other oxidative metabolites as part of metabolomics panel.

Brain MRI/MRS: Magnetic resonance imaging and spectroscopy will be performed using standard neuroimaging procedures.

EEG: Electroencephalography (EEG) will be performed using standard clinical techniques.

Transepidermal water loss (TEWL): TEWL is increased in SLS subjects by up to 90%. TEWL measurements will be obtained using a Tewameter TM300 instrument (Courage+Khazaka Electronic, Inc). This noninvasive procedure measures water vapor loss using a surface skin probe. Three separate measurements will be obtained over a period of 60 seconds each, and the mean TEWL will be calculated as water loss (g/m²/hr) and recorded. In SLS subjects (n=12), the method has a mean analytical CV of 5.4% for the forearm and CV 14.5% for the leg.

Cutaneous measurements: Skin thickness and elasticity will be measured using high-frequency Ultrasound with Shear-wave Elastography (US-SWE). This non-invasive method provides a precise quantitative measure of skin thickness, which is increased in SLS, and reveals a diminished quantitative measure of skin elastography. Ultrasound images and data will be collected with the Aplio 900 (Canon Medical Systems USA, Inc) with a PLT-1204BT multi-frequency linear transducer. Skin thickness will be measured 3 times at the same location, averaged, and expressed in millimeters. SWE measurements provide a mean and SD of elastic modulus values in kilopascals. All measurements will be made from the mid-forearm and mid-anterior thigh where TEWL will also be measured.

Visual Index of Ichthyosis Severity (VIIS): The VIIS is a clinical scoring system that uses 4 body sites³⁷. Photographs of the skin will be taken using a high-resolution digital camera and coded for subsequent analysis. Photographs will be scored in an anonymous fashion by 3 independent observers for the severity of ichthyosis and erythema. The VIIS generates numerically discrete values from 0-4, and the mean score of the 3 observers will be recorded.

Ophthalmologic examination: Visual acuity, retinal photographs and optical coherence tomography (OCT) will be performed. Retinal photographs will be submitted to image analysis to document changes in the pattern and density of crystalline inclusions as described¹². OCT is a routine non-invasive imaging test that uses light waves to generate cross-section images of the retina. SLS subjects exhibit macular degeneration and retinal thinning on OCT^{9,10}.

### Subject (Caregiver) Assessments

At Baseline and Week-12, subjects or their caregivers will take self-reported assessments of overall symptoms on a 6-point scale (0=totally improved to 5=worse) and treatment satisfaction on a 4-point scale (1=excellent to 4=poor). Similarly, assessments of neurologic symptoms and ichthyosis will also be assessed individually.

### REFERENCES

1. W B R. Sjögren-Larsson syndrome: fatty aldehyde dehydrogenase deficiency. The metabolic & molecular bases of inherited disease. New York: McGraw-Hill; 2001:2239-2258.
2. Rizzo W B. Sjögren-Larsson syndrome: molecular genetics and biochemical pathogenesis of fatty aldehyde dehydrogenase deficiency. Mol Genet Metab. 2007; 90: 1-9.
3. Fuijkschot J, Theelen T, Seyger M M B et al. Sjögren-Larsson syndrome in clinical practice. J Inherit Metab Dis. 2012; 35: 955-962.
4. Sjögren T Larsson T. Oligophrenia in combination with congenital ichthyosis and spastic disorders. Acta Psychiatr Neurol Scand. 1957; 32: 1-113.
5. Theile U. Sjögren-Larsson syndrome. Oligophrenia-ichthyosis-di-tetraplegia Humangenetik. 1974; 22: 91-118.
6. Rizzo W B, Jenkens S M, Boucher P. Recognition and diagnosis of neuro-ichthyotic syndromes. Semin Neurol. 2012; 32: 75-84.
7. Jagell S, Lidén S. Ichthyosis in the Sjögren-Larsson syndrome. Clin Genet. 1982; 21: 243-252.
8. Jagell S, Heijbel J. Sjögren-Larsson syndrome: physical and neurological features. A survey of 35 patients. Helv Paediatr Acta. 1982; 37: 519-530.
9. Fouzdar-Jain S, Suh D W, Rizzo W B. Sjögren-Larsson syndrome: a complex metabolic disease with a distinctive ocular phenotype. Ophthalmic Genet. 2019; 40: 298-308.
10. Staps P, Cruysberg J R M, Roeleveld N, Willemsen M A A P, Theelen T. Retinal Morphology in Sjögren-Larsson Syndrome on OCT: From metabolic crystalline maculopathy to early-onset macular degeneration. Ophthalmol Retina. 2019; 3: 500-509.
11. Willemsen M A, Cruysberg J R, Rotteveel J J, Aandekerk A L, Van Domburg P H, Deutman A F. Juvenile macular dystrophy associated with deficient activity of fatty aldehyde dehydrogenase in Sjögren-Larsson syndrome. Am J Ophthalmol. 2000; 130: 782-789.
12. Al-Holou S N, Siefker E, Fouzdar-Jain S, Suh D W, Rizzo W B. Macular crystalline inclusions in Sjögren-Larsson syndrome are dynamic structures that undergo remodeling. Ophthalmic Genet. 2020; 41: 381-385.
13. Warrack S, Love T, Rizzo W B. A neurodegenerative phenotype associated with Sjögren-Larsson syndrome. J Child Neurol. 2021; DOI: 10.1177/08830738211029390.
14. Staps P, van Gaalen J, van Domburg P et al. Sjögren-Larsson syndrome: The mild end of the phenotypic spectrum. JIMD Rep. 2020; 53: 61-70.
15. De Laurenzi V, Rogers G R, Hamrock D J et al. Sjögren-Larsson syndrome is caused by mutations in the fatty aldehyde dehydrogenase gene. Nat Genet. 1996; 12: 52-57.
16. Rizzo W B, Carney G, Lin Z. The molecular basis of Sjögren-Larsson syndrome: mutation analysis of the fatty aldehyde dehydrogenase gene. Am J Hum Genet. 1999; 65: 1547-1560.
17. Weustenfeld M, Eidelpes R, Schmuth M, Rizzo W B, Zschocke J, Keller M A. Genotype and phenotype variability in Sjögren-Larsson syndrome. Hum Mutat. 2019; 40:177-186.
18. Rizzo W B, Craft D A. Sjögren-Larsson syndrome. Deficient activity of the fatty aldehyde dehydrogenase component of fatty alcohol:NAD⁺ oxidoreductase in cultured fibroblasts. J Clin Invest. 1991; 88: 1643-1648.
19. Rizzo W B, Dammann A L, Craft D A. Sjögren-Larsson syndrome. Impaired fatty alcohol oxidation in cultured fibroblasts due to deficient fatty alcohol:nicotinamide adenine dinucleotide oxidoreductase activity. J Clin Invest. 1988; 81: 738-744.
20. Rizzo W B. Fatty aldehyde and fatty alcohol metabolism: review and importance for epidermal structure and function. Biochim Biophys Acta. 2014; 1841: 377-389.
21. Rizzo W B. Genetics and prospective therapeutic targets for Sjögren-Larsson syndrome. Expert Opin Orphan Drugs. 2016; 4: 395-406.
22. Keller M A, Watschinger K, Lange K et al. Studying fatty aldehyde metabolism in living cells with pyrene-labeled compounds. J Lipid Res. 2012; 53: 1410-1416.
23. Rizzo W B, Craft D A. Sjögren-Larsson syndrome: accumulation of free fatty alcohols in cultured fibroblasts and plasma. J Lipid Res. 2000; 41: 1077-1081.
24. S'aulis D, Khoury E A, Zabel M, Rizzo W B. 1-O-Alkylglycerol accumulation reveals abnormal ether glycerolipid metabolism in Sjögren-Larsson syndrome. Mol Genet Metab. 2020; 131: 253-258.
25. Staps P, Rizzo W B, Vaz F M et al. Disturbed brain ether lipid metabolism and histology in Sjögren-Larsson syndrome. J Inherit Metab Dis. 2020; 43: 1265-1278.
26. Rizzo W B, Craft D A, Somer T. Carney G, Trafrova J, Simon M. Abnormal fatty alcohol metabolism in cultured keratinocytes from patients with Sjögren-Larsson syndrome. J Lipid Res. 2008; 49: 410-419.
27. Rizzo W B, S'Aulis D, Jennings M A, Crumrine D A, Williams M L, Elias P M. Ichthyosis in Sjögren-Larsson syndrome reflects defective barrier function due to abnormal lamellar body structure and secretion. Arch Dermatol Res. 2010; 302: 443-451.
28. Nakayama M, Tivora D G F, Alvim T C L, Araijo A C B, Gama R L. MRI and 1H-MRS findings of three patients with Sjögren-Larsson syndrome. Arq Neuropsiquiatr. 2006; 64: 398-401.
29. van Domburg P H, Willemsen M A, Rotteveel J J et al. Sjögren-Larsson syndrome: clinical and MRI/MRS findings in FALDH-deficient patients. Neurology. 1999; 52: 1345-1352.
30. Willemsen M A A P, Van Der Graaf M, Van Der Knaap M S et al. MR imaging and proton MR spectroscopic studies in Sjögren-Larsson syndrome: characterization of the leukoencephalopathy. AJNR Am J Neuroradiol. 2004; 25: 649-657.
31. Hidalgo E T, Orillac C, Hersh A, Harter D H, Rizzo W B, Weiner HL. Intrathecal Baclofen therapy for the treatment of spasticity in Sjögren-Larsson syndrome. J Child Neural. 2017; 32: 100-103.
32. Maaswinkel-Mooij P D, Brouwer O F, Rizzo W B. Unsuccessful dietary treatment of Sjögren-Larsson syndrome. J Pediatr. 1994: 124: 748-750.
33. Willemsen M A, de Jong J G, van Domburg P H, Rotteveel J J, Wanders R J, Mayatepek E. Defective inactivation of leukotriene B4 in patients with Sjögren-Larsson syndrome. J Pediatr. 2000; 136: 258-260.
34. James P F, Rizzo W B, Lee J, Zoeller R A. Isolation and characterization of a Chinese hamster ovary cell line deficient in fatty alcohol:NAD+ oxidoreductase activity. Proc Natl Acad Sci USA. 1990; 87: 6102-6106.
35. Rizzo W B, Bailey Z, S'Aulis D, Ergasheva N. Aldehyde trapping agent NS2 blocks formation of fatty aldehyde adducts with phosphatidylethanolamine and suggests potential therapeutic approach for Sjögren-Larsson syndrome. Mol Genet Metab. 2015; 114: 362-362.
36. Jacques P F, Bostom A G, Wilson P W, Rich S, Rosenberg I H, Selhub J. Determinants of plasma total homocysteine concentration in the Framingham Offspring cohort. Am J Clin Nutr. 2001; 73: 613-621.
37. Marukian N V, Deng Y, Gan G et al. Establishing and Validating an Ichthyosis Severity Index. J Invest Dermatol. 2017; 137: 1834-1841.
38. Bykerk V P. Patient-Reported Outcomes Measurement Information System Versus Legacy Instruments: Are They Ready for Prime Time. Rheum Dis Clin North Am. 2019; 45: 211-229.
39. Dreyfus I, Taïeb C, Barbarot S et al. IQoL-32: a new ichthyosis-specific measure of quality of life. J Am Acad Dermatol. 2013; 69: 82-87.
40. Liu W, Xu L, Lamberson C, Haas D, Korade Z, Porter N A. A highly sensitive method for analysis of 7-dehydrocholesterol for the study of Smith-Lemli-Opitz syndrome. J Lipid Res. 2014; 55: 329-337.

We claim:

1. A method of treating a disease, disorder, or condition selected from hidradenitis suppurativa, toxoplasmosis, Sjögren-Larsson syndrome (SLS), Severe Premenstrual Syndrome (severe PMS), premenstrual dysphoric disorder (PMDD), obsessive-compulsive disorder (OCD), bipolar disorder, depression, major depressive disorder (MDD), or Posttraumatic Stress Disorder (PTSD), comprising administering to a subject in need thereof an effective amount of Compound 1: 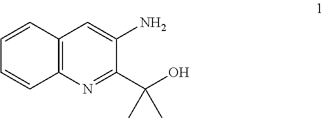
or a pharmaceutically acceptable salt thereof.

2. A method of treating Sjögren-Larsson syndrome (SLS), comprising administering to a patient in need thereof an effective amount of Compound 1: 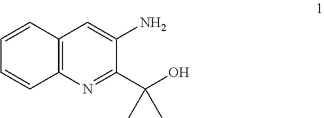
or a pharmaceutically acceptable salt thereof.

3. The method of claim 1 or 2, wherein Compound 1 or a pharmaceutically acceptable salt thereof is administered at a dose of about 100 mg to about 600 mg per day.

4. The method of claim 1 or 2, wherein Compound 1 or a pharmaceutically acceptable salt thereof is administered at a dose of about 250 mg per day.

5. The method of claim 2, wherein Compound 1 or a pharmaceutically acceptable salt thereof is administered systemically at a dose of about 100 mg to about 600 mg per day.

6. The method of claim 5, wherein Compound 1 or a pharmaceutically acceptable salt thereof is administered orally.

7. The method of claim 5 or 6, wherein the method improves pruritis or ichthyosis.

8. The method of any one of claims 5-7, wherein the method improves myopia, photophobia, or macular degeneration.

9. The method of any one of claims 5-8, wherein the method improves seizures, spasticity, cognitive delay, neurological symptoms, or neurodegeneration associated with SLS.

10. A method of reducing levels of one or more toxic aldehydes in a subject having Sjögren-Larsson syndrome (SLS), comprising administering to a patient in need thereof an effective amount of Compound 1: 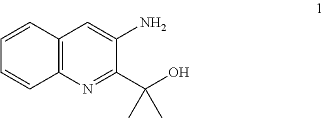
or a pharmaceutically acceptable salt thereof.

11. The method of claim 10, wherein the toxic aldehyde is selected from formaldehyde, acetaldehyde, acrolein, glyoxal, methylglyoxal, hexadecanal, octadecanal, hexadecenal, succinic semi-aldehyde, malondialdehyde, 4-hydroxynonenal, 4-hydroxy-2E-hexenal, 4-hydroxy-2E,6Z-dodecadienal, retinaldehyde, leukotriene B4 aldehyde, octadecenal, and long chain or very long chain (VLC) fatty aldehydes.

12. The method of claim 11, wherein the toxic aldehyde is selected from C17-C20 long chain fatty aldehydes and C21-C24 VLC fatty aldehydes.

13. The method of any one of claims 10-12, wherein Compound 1 or a pharmaceutically acceptable salt thereof is administered at a dose of about 100 mg to about 600 mg per day.

14. The method of any one of claims 10-13, wherein Compound 1 or a pharmaceutically acceptable salt thereof is administered at a dose of about 250 mg per day.

15. The method of any one of claims 10-14, wherein Compound 1 or a pharmaceutically acceptable salt thereof is administered systemically.

16. The method of any one of claims 10-15, wherein Compound 1 or a pharmaceutically acceptable salt thereof is administered orally.
